SEQUENCE LISTING (11 sequences)

SEQ ID NO: 1 - DNA, 1376 bp, Homo sapiens
gtaagaacactctcgtgagtctaacggtcttccggatgaaggctatttgaagtcgccataacctggtcagaagtgtgcctgtcggcggggagagaggcaatatcaaggttttaaatctcggagaaatggctttcgtttgcttggctatcggatgcttatatacctttctgataagcacaacatttggctgtacttcatcttcagacaccgagataaaagttaaccctcctcaggattttgagatagtggatcccggatacttaggttatctctatttgcaatggcaacccccactgtctctggatcattttaaggaatgcacagtggaatatgaactaaaataccgaaacattggtagtgaaacatggaagaccatcattactaagaatctacattacaaagatgggtttgatcttaacaagggcattgaagcgaagatacacacgcttttaccatggcaatgcacaaatggatcagaagttcaaagttcctgggcagaaactacttattggatatcaccacaaggaattccagaaactaaagttcaggatatggattgcgtatattacaattggcaatatttactctgttcttggaaacctggcataggtgtacttcttgataccaattacaacttgttttactggtatgagggcttggatcatgcattacagtgtgttgattacatcaaggctgatggacaaaatataggatgcagatttccctatttggaggcatcagactataaagatttctatatttgtgttaatggatcatcagagaacaagcctatcagatccagttatttcacttttcagcttcaaaatatagttaaacctttgccgccagtctatcttacttttactcgggagagttcatgtgaaattaagctgaaatggagcatacctttgggacctattccagcaaggtgttttgattatgaaattgagatcagagaagatgatactaccttggtgactgctacagttgaaaatgaaacatacaccttgaaaacaacaaatgaaacccgacaattatgctttgtagtaagaagcaaagtgaatatttattgctcagatgacggaatttggagtgagtggagtgataaacaatgctgggaaggtgaagacctatcgaagaaaactttgctacgtttctggctaccatttggtttcatcttaatattagttatatttgtaaccggtctgcttttgcgtaagccaaacacctacccaaaaatgattccagaatttttctgtgatacatgaagactttccatatcaagagacatggtattgactcaacagtttccagtcatggccaaatgttcaatatgagtctcaataaactgaatttttcttgcgaatgttgaaaaa

SEQ ID NO: 2 - protein, 380 aa, Homo Sapiens
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 3 - DNA, 2354 bp, Homo Sapiens
tttttttcccctgctctcccaggggccagacaccaccgccccacccctcacgccccacctccctgggggatcctttccgccccagccctgaaagcgttaaccctggagctttctgcacaccccccgaccgctcccgcccaagcttcctaaaaaagaaaggtgcaaagtttggtccaggatagaaaaatgactgatcaaaggcaggcgatacttcctgttgccgggacgctatatataacgtgatgagcgcacgggctgcggagacgcaccggagcgctcgcccagccgccgcctccaagcccctgaggtttccggggaccacaatgaacaacttgctgtgctgcgcgctcgtgtttctggacatctccattaagtggaccacccaggaaacgtttcctccaaagtaccttcattatgacgaagaaacctctcatcagctgttgtgtgacaaatgtcctcctggtacctacctaaaacaacactgtacagcaaagtggaagaccgtgtgcgccccttgccctgaccactactacacagacagctggcacaccagtgacgagtgtctatactgcagccccgtgtgcaaggagctgcagtacgtcaagcaggagtgcaatcgcacccacaaccgcgtgtgcgaatgcaaggaagggcgctaccttgagatagagttctgcttgaaacataggagctgccctcctggatttggagtggtgcaagctggaaccccagagcgaaatacagtttgcaaaagatgtccagatgggttcttctcaaatgagacgtcatctaaagcaccctgtagaaaacacacaaattgcagtgtctttggtctcctgctaactcagaaaggaaatgcaacacacgacaacatatgttccggaaacagtgaatcaactcaaaaatgtggaatagatgttaccctgtgtgaggaggcattcttcaggtttgctgttcctacaaagtttacgcctaactggcttagtgtcttggtagacaatttgcctggcaccaaagtaaacgcagagagtgtagagaggataaaacggcaacacagctcacaagaacagactttccagctgctgaagttatggaaacatcaaaacaaagaccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggcgaataaaaaatggcgaccaagacaccttgaagggcctaatgcacgcactaaagcactcaaagacgtaccactttcccaaaactgtcactcagagtctaaagaagaccatcaggttccttcacagcttcacaatgtacaaattgtatcagaagttatttttagaaatgataggtaaccaggtccaatcagtaaaaataagctgcttataactggaaatggccattgagctgtttcctcacaattggcgagatcccatggatgagtaaactgtttctcaggcacttgaggctttcagtgatatctttctcattaccagtgactaattttgccacagggtactaaaagaaactatgatgtggagaaaggactaacatctcctccaataaaccccaaatggttaatccaactgtcagatctggatcgttatctactgactatattttcccttattactgcttgcagtaattcaactggaaattaaaaaaaaaaaactagactccattgtgccttactaaatatgggaatgtctaacttaaatagctttgagatttcagctatgctagaggcttttattagaaagccatatttttttctgtaaaagttactaatatatctgtaacactattacagtattgctatttatattcattcagatataagatttgtacatattatcatcctataaagaaacggtatgacttaattttagaaagaaaattatattctgtttattatgacaaatgaaagagaaaatatatatttttaatggaaagtttgtagcatttttctaataggtactgccatatttttctgtgtggagtatttttataattttatctgtataagctgtaatatcattttatagaaaatgcattatttagtcaattgtttaatgttggaaaacatatgaaatataaattatctgaatattagatgctctgagaaattgaatgtaccttatttaaaagattttatggttttataactatataaatgacattattaaagttttcaaattattttttaaaaaaaaa

SEQ ID NO: 4 - protein, 401 aa, Homo Sapiens
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 5 - DNA, 3897 bp, Homo Sapiens
cagtttgcaaaagccagaggtgcaagaagcagcgactgcagcagcagcagcagcagcggcggtggcagcagcagcagcagcggcggcagcagcagcagcagcggaggcaccggtggcagcagcagcatcaccagcaacaacaacaaaaaaaaatcctcatcaaatcctcacctaagctttcagtgtatccagatccacatcttcactcaagccaggagagggaaagaggaaaggggggcaggaaaaaaaaaaaacccaacaacttagcggaaacttctcagagaatgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataaccagggagaggttattcacaacctcaccaaactagtatcattttaggggtgttgacacaccagttttgagtgtactgtgcctggtttgatttttttaaagtagttcctattttctatcccccttaaagaaaattgcatgaaactaggcttctgtaatcaatatcccaacattctgcaatggcagcattcccaccaacaaaatccatgtgaccattctgcctctcctcaggagaaagtaccctcttttaccaacttcctctgccatgtttttcccctgctcccctgagaccacccccaaacacaaaacattcatgtaactctccagccattgtaatttgaagatgtggatccctttagaacggttgccccagtagagttagctgataaggaaactttatttaaatgcatgtcttaaatgctcataaagatgttaaatggaattcgtgttatgaatctgtgctggccatggacgaatatgaatgtcacatttgaattcttgatctctaatgagctagtgtcttatggtcttgatcctccaatgtctaattttctttccgacacatttaccaaattgcttgagcctggctgtccaaccagactttgagcctgcatcttcttgcatctaatgaaaaacaaaaagctaacatctttacgtactgtaactgctcagagctttaaaagtatctttaacaattgtcttaaaaccagagaatcttaaggtctaactgtggaatataaatagctgaaaactaatgtactgtacataaattccagaggactctgcttaaacaaagcagtatataataactttattgcatatagatttagttttgtaacttagctttatttttcttttcctgggaatggaataactatctcacttccagatatccacataaatgctccttgtggccttttttataactaagggggtagaagtagttttaattcaacatcaaaacttaagatgggcctgtatgagacaggaaaaaccaacaggtttatctgaaggaccccaggtaagatgttaatctcccagcccacctcaacccagaggctactcttgacttagacctatactgaaagatctctgtcacatccaactggaaattccaggaaccaaaaagagcatccctatgggcttggaccacttacagtgtgataaggcctactatacattaggaagtggcagttctttactcgtcccctttcatcggtgcctggtactctggcaaatgatgatggggtgggagactttccattaaatcaatcaggaatgagtcaatcagcctttaggtctttagtccgggggacttggggctgagagagtataaataaccctgggctgtccagccttaatagacttctcttacattttcgtcctgtagcacgctgcctgccaaagtagtcctggcagctggaccatctctgtaggatcgtaaaaaaatagaaaaaaagaaaaaaaaaagaaagaaagagggaaaaagagctggtggtttgatcatttctgccatgatgtttacaagatggcgaccaccaaagtcaaacgactaacctatctatgaacaacagtagtttctcagggtcactgtccttgaacccaacagtcccttatgagcgtcactgcccaccaaaggtcaatgtcaagagaggaagagagggaggaggggtaggactgcaggggccactccaaactcgcttaggtagaaactattggtgcttgactctcactaggctaaactcaagatttgaccaaatcgagtgatagggatcctggtgggaggagagagggcacatctccagaaaaatgaaaagcaatacaactttaccataaagcctttaaaaccagtaacgtgctgctcaaggaccaagagcaattgcagcagacccagcagcagcagcagcagcacaaacattgctgcctttgtccccacacagcctctaagcgtgctgacatcagattgttaagggcatttttatactcagaactgtcccatccccaggtccccaaacttatggacactgccttagcctcttggaaatcaggtagaccatattctaagttagactcttcccctccctcccacacttcccacccccaggcaaggctgacttctctgaatcagaaaagctattaaagtttgtgtgttgtgtccattttgcaaacccaactaagccaggaccccaatgcgacaagtagttcatgagtattcctagcaaatttctctctttcttcagttcagtagatttccttttttcttttctttttttttttttttttttttggctgtgacctcttcaaaccgtggtacccccccttttctccccacgatgatatctatatatgtatctacaatacatatatctacacatacagaaagaagcagttctcacaatgttgctagttttttgcttctctttcccccaccctactccctccaattcccccttaaacttccaaagcttcgtcttgtgtttgctgcagagtgattcgggggctgacctagaccagtttgcatgattcttctcttgtgatttggttgcactttagacatttttgtgccattatatttgcattatgtatttataatttaaatgatatttaggtttttggctgagtactggaataaacagtgagcatatctggtatatgtcattatttattgttaaattacatttttaagctccatgtgcatataaaggttatgaaacatatcatggtaatgacagatgcaagttattttatttgcttatttttataattaaagatgccatagcataatatgaagcctttggtgaattccttctaagataaaaataataataaagtgttacgttttattggtttcaaaaaaaaaaaaaaaaaaaa

SEQ ID NO: 6 - protein, 247 aa, Homo Sapiens
MLQNSAVLLVLVISASATHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 7 - DNA, 4507 bp, Homo Sapiens
gaccaattgtcatacgacttgcagtgagcgtcaggagcacgtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtgttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactgtcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagtagaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatcttaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataatgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatatcttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactatttaaaaattgacagatcttttctgaagaaaaaaaaaaaaaaa

SEQ ID NO: 8 - protein, 604 aa, Homo Sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 9 - DNA, 2354 bp, Homo Sapiens
gctcagggcacatgcctcccctccccaggccgcggcccagctgaccctcggggctcccccggcagcggacagggaagggttaaaggcccccggctccctgccccctgccctggggaacccctggccctgtggggacatgaactgtgtttgccgcctggtcctggtcgtgctgagcctgtggccagatacagctgtcgcccctgggccaccacctggcccccctcgagtttccccagaccctcgggccgagctggacagcaccgtgctcctgacccgctctctcctggcggacacgcggcagctggctgcacagctgagggacaaattcccagctgacggggaccacaacctggattccctgcccaccctggccatgagtgcgggggcactgggagctctacagctcccaggtgtgctgacaaggctgcgagcggacctactgtcctacctgcggcacgtgcagtggctgcgccgggcaggtggctcttccctgaagaccctggagcccgagctgggcaccctgcaggcccgactggaccggctgctgcgccggctgcagctcctgatgtcccgcctggccctgccccagccacccccggacccgccggcgcccccgctggcgcccccctcctcagcctgggggggcatcagggccgcccacgccatcctgggggggctgcacctgacacttgactgggccgtgaggggactgctgctgctgaagactcggctgtgacccggggcccaaagccaccaccgtccttccaaagccagatcttatttatttatttatttcagtactgggggcgaaacagccaggtgatccccccgccattatctccccctagttagagacagtccttccgtgaggcctggggggcatctgtgccttatttatacttatttatttcaggagcaggggtgggaggcaggtggactcctgggtccccgaggaggaggggactggggtcccggattcttgggtctccaagaagtctgtccacagacttctgccctggctcttccccatctaggcctgggcaggaacatatattatttatttaagcaattacttttcatgttggggtggggacggaggggaaagggaagcctgggtttttgtacaaaaatgtgagaaacctttgtgagacagagaacagggaattaaatgtgtcatacatatccacttgagggcgatttgtctgagagctggggctggatgcttgggtaactggggcagggcaggtggaggggagacctccattcaggtggaggtcccgagtgggcggggcagcgactgggagatgggtcggtcacccagacagctctgtggaggcagggtctgagccttgcctggggccccgcactgcatagggccttttgtttgttttttgagatggagtctcgctctgttgcctaggctggagtgcagtgaggcaatctgaggtcactgcaacctccacctcccgggttcaagcaattctcctgcctcagcctcccgattagctgggatcacaggtgtgcaccaccatgcccagctaattatttatttcttttgtatttttagtagagacagggtttcaccatgttggccaggctggtttcgaactcctgacctcaggtgatcctcctgcctcggcctcccaaagtgctgggattacaggtgtgagccaccacacctgacccataggtcttcaataaatatttaatggaaggttccacaagtcaccctgtgatcaacagtacccgtatgggacaaagctgcaaggtcaagatggttcattatggctgtgttcaccatagcaaactggaaacaatctagatatccaacagtgagggttaagcaacatggtgcatctgtggatagaacgccacccagccgcccggagcagggactgtcattcagggaggctaaggagagaggcttgcttgggatatagaaagatatcctgacattggccaggcatggtggctcacgcctgtaatcctggcactttgggaggacgaagcgagtggatcactgaagtccaagagttcgagaccggcctgcgagacatggcaaaaccctgtctcaaaaaagaaagaatgatgtcctgacatgaaacagcaggctacaaaaccactgcatgctgtgatcccaattttgtgtttttctttctatatatggattaaaacaaaaatcctaaagggaaatacgccaaaatgttgacaatgactgtctccaggtcaaaggagagaggtgggattgtgggtgacttttaatgtgtatgattgtctgtattttacagaatttctgccatgactgtgtattttgcatgacacattttaaaaataataaacactatttttagaat

SEQ ID NO: 10 - protein, 199 aa, Homo Sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 11 - protein, 9 aa, Homo Sapiens
AKTTPKLGG

(19) United States
(12) Patent Application Publication
(10) Pub. No.: US 2011/0059445 A1
(43) Pub. Date: Mar. 10, 2011

(54) MUCOSAL GENE SIGNATURES

(76) Inventors: Paul Rutgeerts, Blanden (BE); Frans Schuit, Keerbergen (BE)

(21) Appl. No.: 12/736,759

(22) PCT Filed: Mar. 30, 2009
(86) PCT No.: PCT/BE2009/000021

§ 371 Date: Nov. 5, 2010

Related U.S. Application Data
(60) Provisional application No. 61/072,200, filed on Mar. 28, 2008.

(51) Int. Cl. C12Q 1/68; G01N 33/567

(52) U.S. Cl. 435/6; 435/7.21

(57) ABSTRACT

Infliximab (IFX) is an effective treatment for Crohn's disease (CD) and ulcerative colitis (UC) not responding to standard therapy. Thirty percent to forty percent of patients however do not improve and the response is often incomplete. We identified mucosal gene signatures predictive of response to EFX using high-density oligonucleotide arrays. Eight UC patients and twelve CD patients showed healing. In UC, only one probe set was differentially expressed in responders compared with non-responders, i.e., IL-13R(alpha)2. At PAM analysis, two probe sets, representing IL-13Ralpha2 and IL-I 1, separated IBD responders from non-responders with an overall misclassification error rate of 0.046 (2/43), with 100% sensitivity and 91.3% specificity. The IL-13R(alpha)2 probe set was a top-ranked probe set in all our analyses using both LIMMA and PAM strategies. Our gene array studies of mucosal biopsies identified IL-13R(alpha)2 in IBD as a predictor of response or non-response to IFX.

### CROSS-REFERENCE TO RELATED APPLICATIONS

This is a national phase entry under 35 U.S.C. §371 of International Patent Application PCT/BE2009/000021, filed Mar. 30, 2009, published in English as International Patent Publication WO 2009/117791 A2 on Oct. 1, 2009, which claims the benefit under Article 8 of the Patent Cooperation Treaty to U.S. Provisional Patent Application Ser. No. 61/072,200, filed Mar. 28, 2008.

### STATEMENT ACCORDING TO 37 C.F.R. §1.52(e)(5)—SEQUENCE LISTING SUBMITTED ON COMPACT DISC

Pursuant to 37 C.F.R. §1.52(e)(1)(ii), a compact disc containing an electronic version of the Sequence Listing has been submitted concomitant with this application, the contents of which are hereby incorporated herein by reference. A second compact disc is submitted and is an identical copy of the first compact disc. The discs are labeled “copy 1” and “copy 2,” respectively, and each disc contains one file entitled “2008025-PCT-sequentielijst_ST25.txt” which is 35 KB and created on Nov. 4, 2010.

### TECHNICAL FIELD

The present invention concerns mucosal gene signatures to predict response to an anti-TNFα therapy, such as, for instance, infliximab in patients with inflammatory bowel disease (IBD).

### BACKGROUND

Ulcerative colitis (UC) is a chronic inflammatory bowel disease involving the mucosa of the colon distal to the anal verge. The pathogenesis of UC is believed to be a result of an interaction of genetic factors, the immune response to microbial dysbiosis and environmental factors. Cigarette smoking and appendectomy have both been associated with a decreased risk of developing UC.⁽¹⁾ Five aminosalicylates, corticosteroids and azathiopurine are the current treatments for UC and patients who fail these treatments were, until recently, referred for colectomy. The ACT trials have shown that Infliximab (REMICADE®; Centocor, Inc., Malvern, Pa., USA), a mouse/human chimeric monoclonal IgG1 antibody to tumor necrosis factor alpha (TNFα), is efficacious in the treatment of patients with refractory UC and may avoid colectomy.⁽²⁾ However, around 40% of patients treated do not respond to Infliximab and predictors of response are currently lacking.

Microarray technology is a powerful tool that enables the measurement of the expression of thousands of genes simultaneously.⁽³⁾ This technology has been used to elucidate the pathogenic processes underlying different diseases and to identify predictive gene profiles.^{(4, 5)}

### DISCLOSURE

An initial aim hereof was to identify mucosal gene signatures predictive of response to anti-TNFα therapeutic antibodies, such as, for instance, Infliximab, Adalimumab or Etanercept in anti-TNFα-naive UC patients using high-density oligonucleotide arrays. We studied two independent cohorts of patients, one at the University Hospital Leuven (cohort A) and one cohort of patients who took part in the placebo-controlled ACT1 study⁽²⁾ (cohort B). Infliximab (IFX) is one of such anti-TNFα therapeutic antibodies that is an effective treatment for Crohn's disease (CD) and ulcerative colitis (UC) not responding to standard therapy. Thirty to forty percent of patients do not improve and response is often incomplete. Since the mechanism of resistance to anti-TNFα is unknown, the aim of the study leading to the present invention was to identify mucosal gene signatures predictive of response to IFX using high-density oligonucleotide arrays. This study used colonic mucosal gene expression to provide a predictive response signature for Infliximab treatment in IBD, where under UC and CD.

Gene array studies of UC mucosal biopsies identified predictive panels of genes for (non-) response to an anti-TNFα treatment of inflammatory bowel disease (IBD) such as Crohn's disease (CD) or ulcerative colitis (UC), or for use in monitoring the effectiveness of therapy of inflammatory bowel disease (IBD) such as Crohn's disease (CD) or ulcerative colitis (UC) in patients. This is particularly suitable for predicting the effectiveness of therapy with anti-TNFα therapeutic antibodies, such as, for instance, Infliximab, Adalimumab or Etanercept. Moreover, our gene array studies of mucosal biopsies identified IL-13R(alpha)2 in IBD as a predictor of (non-)response to a therapy of blocking tumor necrosis factor alpha (TNFα), for instance, to IFX. A combined therapy of compound that blocks the action of TNFα by preventing it from binding to its receptor in the cell (such as Infliximab, Adalimumab or Etanercept) and a compound that inhibits the expression of IL-13R(alpha)2 or the activity of expression product is a particular embodiment of the present invention.

Crohn's disease (CD) and ulcerative colitis (UC) are complex and heterogeneous inflammatory bowel diseases (IBD), resulting from an interplay of immune, genetic and environmental factors. New insights into the pathogenesis of IBD have led to the development of biological therapies. Infliximab (REMICADE®; Centocor Inc., Malvern, Pa., USA), a mouse/human chimeric monoclonal IgG1 antibody to tumor necrosis factor alpha (TNFα), was the first clinically available biological therapy for IBD. Infliximab (IFX) has now been used for over seven years for the treatment of refractory luminal and fistulizing CD.⁽¹⁻⁴⁾ The ACT studies have shown that IFX is also efficacious for inducing and maintaining clinical remission and mucosal healing in patients with moderate to severe, active UC who had an inadequate response to standard therapy.⁽⁵⁾ Up to 30% of patients with CD or UC do not respond to this treatment and in 20% to 30%, response is incomplete. IFX is a costly therapy and may be associated with serious side effects. Therefore, it is of critical importance to identify predictors of response to IFX.

Forty-three patients with inflammatory bowel disease (IBD), 19 with Crohn's colitis and 24 with UC, underwent a colonoscopy with biopsies before and 4 to 6 weeks after the first IFX treatment. Response to IFX was defined as endoscopic and histologic healing. Total RNA was isolated from pre-IFX biopsies, labeled and hybridized to AFFYMETRIX® HG U133 plus 2.0 Array. Micorarray data were analyzed using Bioconductor software. Quantitative real-time RT-PCR and immunohistochemistry were used to confirm microarray data. Furthermore, two cohorts of patients who received their first treatment with Infliximab for refractory UC were studied. Response to Infliximab was defined as endoscopic and histologic healing. Total RNA was isolated from colonic mucosal biopsies, labeled and hybridized to AFFYMETRIX® Human Genome U133 Plus 2.0 Arrays. Data were analyzed using Bioconductor software. Quantitative RT-PCR was used to confirm microarray data. Eight UC patients and twelve CD patients showed healing. In UC, only one probe set was differentially expressed in responders compared with non-responders, i.e., IL-13R(alpha)2. At PAM analysis 2 probe sets, representing IL-13R(alpha)2 and IL-11, separated IBD responders from non-responders with an overall misclassification error rate of 0.046 (2/43), with 100% sensitivity and 91.3% specificity. The IL-13R(alpha)2 probe set was a top-ranked probe set in all our analyses using both LIMMA and PAM strategies.

For predicting response to Infliximab treatment, pre-treatment colonic mucosal expression profiles were compared for responders and non-responders. Comparative analysis identified 179 significant probe sets in cohort A and 361 in cohort B with an overlap of 74 probe sets, representing 53 known genes, between both analyses. Comparative analysis of both cohorts combined yielded 212 significant probe sets. The top five significant genes in a combined analysis of both cohorts were osteoprotegerin, stanniocalcin-1, prostaglandin-endoperoxide synthase 2, interleukin-13 receptor alpha 2 and interleukin-11. All proteins encoded by these genes are involved in the adaptive immune response. These markers separated responders from non-responders with 95% sensitivity and 85% specificity.

The present invention is based on the surprising finding that increased IL-13R(alpha)2 expression or IL-13R(alpha)2 activity is suppressive on the response to an anti-TNFα therapy of inflammatory bowel disease (IBD), such as, for instance, Infliximab in patients with inflammatory bowel disease (IBD). This finding indicated that the non-responding to such anti-TNFα therapy of IBD, in particular, UC or CD, can be attenuated by inhibiting the IL-13R(alpha)2 expression or activity. Such interventions have been proposed as a pharmaceutical co-treatment with anti-TNFα therapy of inflammatory bowel disease by the present invention.

Provided is the use of compounds having an inhibitory action on IL-13R(alpha)2 expression or IL-13R(alpha)2 activity in the manufacture of a medicine for the treatment of IBD, in particular, UC or CD.

A first embodiment includes a compound having an inhibitory action on IL-13R(alpha)2 activation or that inhibits the expression and/or activity of IL-13R(alpha)2 for use in a treatment to cure or to prevent IBD, in particular, UC or CD. Such a compound having an inhibitory action on IL-13R(alpha)2 activity or inhibiting the expression and/or activity of IL-13R(alpha)2 can be selected from the group consisting of a nucleotide, an antibody, a ribozyme, and a tetrameric peptide.

The nucleotide to inhibit the expression and/or activity of IL-13R(alpha)2 can be an antisense DNA or RNA, siRNA, miRNA or an RNA aptamer. Other suitable reducing α-synculein activities are the monoclonal antibodies specifically directed to IL-13R(alpha)2 or an antigen-binding fragment thereof. Such an antibody or antibody fragment can be humanized.

A second embodiment of concerns the use of a compound having an inhibitory action on IL-13R(alpha)2 activation or inhibit the expression and/or activity of IL-13R(alpha)2 in the manufacture of a medicament for the co-treatment in anti-TNFα therapy of inflammatory bowel disease to increase the amount of responding patients to such anti-TNFα therapy. IL-13R(alpha)2 antagonists or compounds that inhibit, block or suppress the action of IL-13R(alpha)2 (e.g., expression and/or activity of IL-13R(alpha)2) are available or can be produced with current state-of-the-art technology and are inhibiting nucleotides, antibodies, ribozymes or tetrameric peptides.

### BRIEF DESCRIPTION OF THE DRAWINGS

FIG. 1 provides a hierarchical cluster analysis of R and NR using all DE probe sets in UC (a), CD (b) and IBD (d), as well as the top 20 significantly DE probe sets in CD (c) and IBD (e). Individual samples are shown in columns and genes in rows. The log2 expression values for individual probe sets are indicated by color (in this print, gray scale), as shown in the scale, white yellow (light) indicating a high level of expression and blue (darker) a low level of expression.

FIG. 2 are graphics of the probe set that discriminate R from NR in colonic IBD with an overall ME of 0.046 (2/43), identification by PAM. The y-axis shows the log2 expression values of each patient for the probe sets. Red and blue circled symbols represent NR and R samples, respectively. The dot line represents the separation between R and NR.

FIG. 3 displays expression of IL-13R(alpha)2 in IBD patients before treatment and controls, identified by microarray analysis (a) and quantitative RT-PCR (b). The horizontal lines indicate the average of each group.

FIG. 4 displays IL-13R(alpha)2 immunohistochemistry of (a) an ovarian serous adenocarcinoma, (b) normal colon, (c) ulcerative colitis before IFX treatment, (d) Crohn's colitis before IFX treatment (original magnification, OMs×400 for (a), ×200 for (b), and ×50 for (c) and (d).

FIG. 5 demonstrates a positive linear correlation between the TGF-beta1 probe set 203085_S_at and the IL-13Ralpha2 probe set, based on the log2 expression values from R and NR in IBD before treatment and controls.

FIG. 6 provides the nucleotide and protein sequences of interleukin 13 receptor, alpha 2 (IL-13RA2). FIG. 6A: Homo sapiens interleukin 13 receptor, alpha 2 (IL-13RA2), mRNA (LOCUS NM_{—}000640, 1376 by mRNA linear PRI 17 Feb. 2008) as deposited under accession number NM_{—}000640, version NM_{—}000640.2 GI:26787976. (SEQ ID NO:1) FIG. 6B: IL-13RA2 coding sequences (CDS) 126 . . . 1268. (SEQ ID NO:2)

FIG. 7 provides the nucleotide and protein sequences of TNFRSF11B or tumor necrosis factor receptor superfamily, member 11b. FIG. 7A: Homo sapiens TNFRSF11B, mRNA (LOCUS NM_{—}002546, 2354 by mRNA linear PRI 15 Mar. 2009) as deposited under accession number NM_{—}002546, version NM_{—}002546.3 GI:148743792, gene 1 . . . 2354. (SEQ ID NO:3) FIG. 7B: TNFRSF11B coding sequences (CDS) 324 . . . 1529. (SEQ ID NO:4)

FIG. 8 provides the nucleotide and protein sequences of stanniocalcin 1 or STC1. FIG. 8A: Homo sapiens STC1, mRNA (LOCUS NM_{—}003155 3897 by mRNA linear PRI 29 Mar. 2009) as deposited under accession number NM_{—}003155.2 GI:61676083. (SEQ ID NO:5) FIG. 8B: STC1 coding sequences (CDS) CDS 285 . . . 1028. (SEQ ID NO:6)

FIG. 9 provides the nucleotide and protein sequences of PTGS2 prostaglandin-endoperoxide synthase 2 (prostaglandin G/H synthase and cyclooxygenase). FIG. 9A: Homo sapiens PTGS2, mRNA (LOCUS NM_{—}000963 4507 by mRNA linear PRI 29 Mar. 2009) as deposited under accession number NM_{—}000963 version NM_{—}000963.2 GI:223941909. (SEQ ID NO:7) FIG. 9B: PTGS2 coding sequences (CDS) 138 . . . 1952. (SEQ ID NO:8)

FIG. 10 provides the nucleotide and protein sequences of Homo sapiens interleukin 11 (IL-11). FIG. 10A: Homo sapiens IL-11, mRNA (LOCUS NM_{—}000641 2354 by mRNA linear PRI 29 Mar. 2009) as deposited under accession number NM_{—}000641 version NM_{—}000641.2 GI:24430217. (SEQ ID NO:9) FIG. 10B: IL-11 coding sequences (CDS) 137 . . . 736. (SEQ ID NO:10)

FIG. 11 provides a schematic representation of a diabody expression cassette. The locations of promoter/operator (p/o), rbs, gene encoding Leader 1 and Leader 2 (allowing secretory production of the diabody), Tag 1 (i.e., cMyc epitope) and Tag 2 (i.e., 6HIS, if appropriate) are indicated. Amino acid sequence of the linker (L) connecting the variable domains in each antibody fragment is L=AKTTPKLGG (SEQ ID NO:).

FIG. 12 provides a schematic representation of the assembly of the diabody expression cassette. The locations of promoter/operator (p/o), rbs, gene encoding Leader 1 (L1) and Leader 2 (L2), Tag 1 and Tag 2 are indicated. The sequence of the linker (L) connecting the variable domains in each antibody fragment was incorporated via PCR.

### DETAILED DESCRIPTION OF THE INVENTION

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

Abbreviations in this application are: FC, fold change; FDR, false discovery rate; IL-11, interleukin-11; IL-13R(alpha)2, interleukin-13 receptor alpha 2; IQR, interquartile range; LIMMA, linear models for microarray data; NR, non-responders; PAM, prediction analysis of microarrays; PTGS2, prostaglandin-endoperoxide synthase 2; qPCR, quantitative RT-PCR; R, responders; RMA, robust multichip average; STC1, stanniocalcin-1; TNFα, tumor necrosis factor alpha; TNFRSF11B, osteoprotegerin; UC, ulcerative colitis.

Homo sapiens interleukin 13 receptor alpha 2 (IL-13RA2) has been described in G. P. Katsoulotos et al., J. Biol. Chem. 283 (3), 1610-1621 (2008); T. Tanabe et al., Clin. Exp. Allergy 38 (1), 122-134 (2008); O. Bozinov et al., Neurosurg. Rev. 31 (1), 83-89 (2008); J. S. Jarboe et al., Cancer Res. 67 (17), 7983-7986 (2007); A. L. Andrews et al., J. Allergy Clin. Immunol. 120 (1), 91-97 (2007); D. D. Donaldson et al., J. Immunol. 161 (5), 2317-2324 (1998); J. Guo et al., Chromosome mapping and expression of the human interleukin-13, Genomics 42 (1), 141-145 (1997); J. G. Zhang et al., J. Biol. Chem. 272 (14), 9474-9480 (1997); J. F. Gauchat et al., Eur. J. Immunol. 27 (4), 971-978 (1997); and D. Caput et al., J. Biol. Chem. 271 (28), 16921-16926 (1996); and a sequence for CDR and mRNA has, for instance, been deposited at NCBI under accession NM_{—}000640 (PUBMED 8663118 LOCUS NM_{—}000640 1376 by mRNA linear PRI 17 Feb. 2008).

Infliximab is a therapeutic monoclonal antibody that works by blocking tumor necrosis factor alpha (TNFα, a chemical messenger (cytokine) that is a key part of the autoimmune reaction). Infliximab blocks the action of TNFα by preventing it from binding to its receptor in the cell. Other anti-TNFα therapeutic antibodies are, for instance, Adalimumab (brand name Humira) or Etanercept. Like Infliximab, Etanercept and Adalimumab bind to TNFα, preventing it from activating TNF receptors. The present invention provides a diagnostic method to predict response or non-response in an inflammatory bowel disease (IBD) and, more particularly, ulcerative colitis treatment by compounds that block the action of TNFα, such as Infliximab, Adalimumab or Etanercept.

Forty-three patients with active IBD, 24 UC and 19 CD, refractory to corticosteroids and/or immunosuppression and a control group of six individuals who underwent colonoscopy for screening were studied. Baseline characteristics of the patients are summarized in Table 1. The patients underwent colonoscopy with biopsies from diseased colon within a week prior to the first intravenous infusion of 5 mg IFX per kg body weight. The patients underwent a second colonoscopy four weeks after the first IFX infusion in case of a single infusion and at six weeks if they received a loading dose of IFX at weeks 0, 2 and 6. Part of the biopsies were immediately snap-frozen in liquid nitrogen and stored at −80° C. until RNA isolation. The residual biopsies were fixed in Carnoy's fixative for up to five hours and then dehydrated, cleared and paraffin-embedded for histologic examination.

All patients were followedup long term. The ethics committee of the university hospital approved the study and all individuals gave written informed consent.

Response to IFX was defined as a complete mucosal healing with a decrease of at least three points on the histological score for CD⁽⁹⁾ and as a decrease to a Mayo endoscopic subscore of 0 or 1(5) with a decrease to grade 0 or 1 on the histological score for UC.⁽¹⁰⁾ Patients who did not achieve this healing were considered non-responders although some of them presented improvement.

### Examples

### Example 1

### RNA Isolation:

Total RNA was extracted from the biopsy specimens using the RNEASY® Mini Kit (Qiagen, Benelux B.V.), according to the manufacturer's instructions. The integrity and quantity of total RNA were assessed with the AGILENT® 2100 BIOANALYZER® (Agilent, Waldbronn, Germany) and NANODROP® ND-1000 spectrophotometer (Nanodrop Technologies). The extracted RNA was used for microarray analysis and, in some cases, for quantitative RT-PCR analysis.

### Oligonucleotide Array Hybridization:

All steps were performed according to AFFYMETRIX® expression analysis technical manual 701021Rev.5 (Santa Clara, Calif., USA). Briefly, total RNA (2 μg) was reverse-transcribed into cDNA using the SUPERSCRIPT® Choice System (Invitrogen, Carlsbad, Calif., USA). cDNA was in vitro transcribed to cRNA and biotin labeled (Affymetrix, Santa Clara, Calif., USA). Biotinylated cRNA was purified and fragmented. The quality of labeled and fragmented cRNA, respectively, was assessed with the AGILENT® 2100 BIOANALYZER®. Fragmented cRNA (15 μg) was hybridized overnight to the Human Genome U133 Plus 2.0 Array (Affymetrix, Santa Clara, Calif., USA). The arrays were washed and stained with streptavidin-phycoerytrin and scanned on the AFFYMETRIX® 3000 GeneScanner. The resulting image files (.dat files) were analyzed using AFFYMETRIX® GCOS software, and intensity values for each probe cell (.cel file) were calculated. Quality evaluation of the microarrays were as expected. The data are available at ArrayExpress, a public repository for microarray data (accession number and address).

### Data Analysis:

The AFFYMETRIX® raw data (.cel files) were analyzed using Bioconductor tools⁽¹¹⁾ in R (version 2.4.1, http://r-project.org/). Probe level analysis was performed with the robust multichip average (RMA) method.⁽¹²⁾ Linear models for microarray data (LIMMA)⁽¹³⁾ and prediction analysis of microarrays (PAM)⁽¹⁴⁾ were used for supervised data analyses. LIMMA was used to identify differentially expressed (DE) probe sets between the groups, on the basis of moderated t-statistics using an empirical Bayes method, with the use of false discovery rate (FDR) correction for multiple testing (Benjamini and Hochberg⁽¹⁵⁾). A greater than two-fold change combined with a FDR<0.05 were considered statistically significant. PAM, a nearest shrunken centroid method, was used to identify a subset of probe sets that can be used to classify pre-treatment samples as responder (R) or non-responder (NR). PAM was applied to the entire dataset using leave-one-out cross-validation. Unsupervised hierarchical clustering based on the average-linkage method with the Euclidian distance metric was performed to visualize gene (probe set)/sample relationship. The Functional Annotation tool on the DAVID homepage (http://david.abcc.ncifcrf.gov/home.jsp) was used for Gene Ontology (GO) analysis to find biological processes that are overrepresented among sets of DE probe sets.⁽¹⁶⁾ Only GO categories represented by more than ten probe sets in the set of DE probe sets and an EASE score<0.01 were considered significant.

### Quantitative RT-PCR:

To validate the microarray data, quantitative duplex real-time RT-PCR for the genes IL-13R(alpha)2 and β-actin (the endogenous reference gene) was performed, using total RNA from samples that were used for the microarrays. cDNA was synthesized from 0.5 μg of total RNA using the REVERTAID™ H Minus First Strand cDNAsynthesis kit (Fermentas, St. Leon-Rot, Germany), following the manufacturer's protocol. Primers and dual-labeled probes were designed using OligoAnalyzer 3.0 software (http://biotools.idtdna.com/analyzer/) and synthesized by Sigma-Genosys Ltd. (Haverhill, UK). The oligonucleotide sequences are available upon request. Real-time PCR was performed in a final reaction volume of 25 μl on a ROTOR-GENE® 3000 instrument (Corbett Research Pty Ltd., Mortlake, Australia), using QUANTITECT® Multiplex PCR NoROX Kit (Qiagen, Venlo, NL), according to the manufacturer's instructions. Cycle threshold values were determined by ROTOR-GENE® 6.0.16 software. All samples were amplified in duplicate reactions. The relative expression of target mRNA levels were calculated as a ratio relative to the β-actin reference mRNA.⁽¹⁷⁾ Results were analyzed using the Mann-Whitney U-test using SPSS 15.0 software (SPSS, Chicago, Ill.) and a P-value of <0.05 was considered significant.

### IL-13Ralpha2 Immunohistochemistry:

To determine the IL-13R(alpha)2 protein localization, immunohistochemical staining was performed on 5-μm thick step sections prepared from each paraffin block. Endogenous peroxidase activity was blocked in dewaxed sections by incubating the slides for 20 minutes in a 0.3% solution of H₂O₂ in methanol. Epitope retrieval was performed by heating the slides for 30 minutes in Tris/EDTA buffer (pH 9) at 98° C. Sections were then incubated with the antihuman IL-13R(alpha)2 mouse monoclonal antibody clone ab55275 (Abcam plc, Cambridge, United Kingdom) at a concentration of 1 μg/mL for 30 minutes. The Dako REAL™ Envision™ Detection System kit (Dako Belgium NV, Heverlee, Belgium) was used for visualization of bound primary antibody according to the manufacturer's instructions. Formalin-fixed, paraffin-embedded surgical biopsies of an ovarian serous adenocarcinoma served as positive controls.⁽¹⁸⁾ The primary antibody was omitted in the negative controls.

### Study of the IL-13, IL-13R(Alpha)1, IL-13R(Alpha)2 and TGF-Beta1 Pathway:

In order to identify mechanisms of non-response, we compared the gene intensities for pathway-related molecules including IL-13, IL-13Ralphal, IL-13R(alpha)2 and TGF-beta1 in R and NR and explored the relationships between the probe sets. The correlation between probe sets was analyzed using the Spearman's Rank Correlation test using SPSS 15.0 software.

### Results:

### Identification of Differentially Expressed Probe Sets Between R and NR:

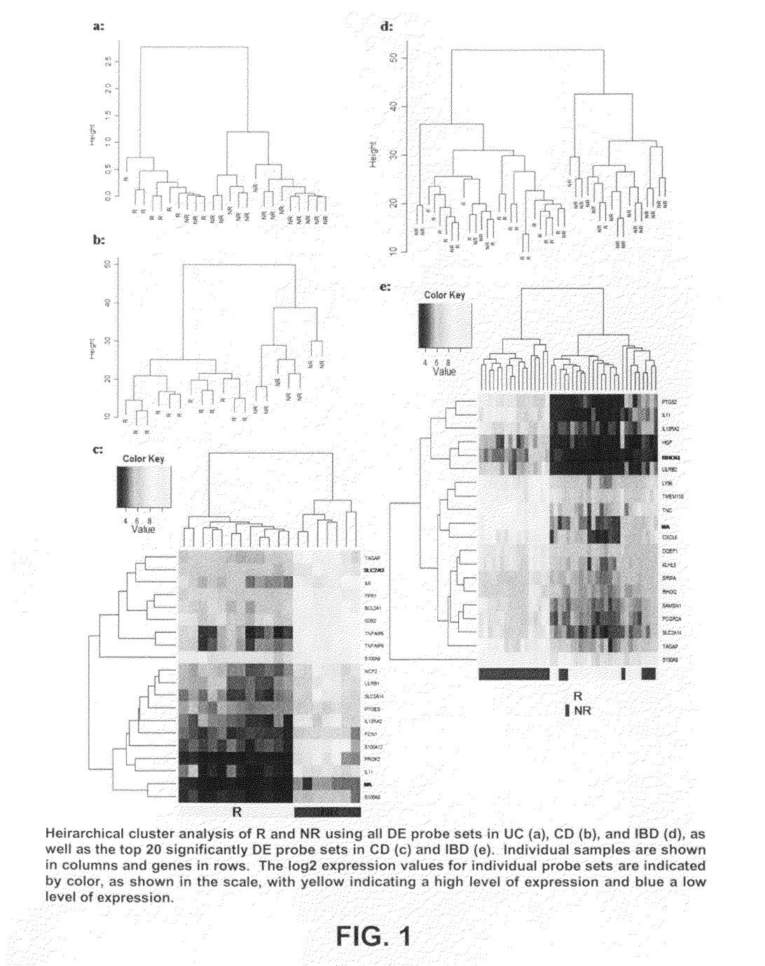

We identified 20 R (8 UC and 12 CD) and 23 NR (16 UC and 7 CD). There were no baseline characteristics predictive of response. For predicting response to IFX treatment based on gene profiles, pre-treatment expression profiles were compared for R and NR, using LIMMA. In UC, only one probe set, representing IL-13R(alpha)2, was DE between R and NR with an up-regulation in NR. Hierarchical clustering, based on the log₂ expression values of IL-13alpha2 from UC patients, resulted in two major clusters of R versus NR, with two NR misclassified in the cluster of R (FIG. 1, Panel a). At LIMMA analysis, a total of 697 probe sets were DE in CD, with five probe sets showing an increased expression and 692 a decreased expression in R compared with NR. Hierarchical cluster analysis using all DE probe sets (FIG. 1, Panel b), as well as the top 20 significantly DE probe sets (FIG. 1, Panel c), completely separated R from NR in CD. For IBD overall (UC and CD), 672 DE probe sets were identified between R and NR. Thirty-one of the 672 DE probe sets showed an increased expression and 641 probe sets showed a decreased expression in R compared to NR. Cluster analysis, using all DE probe sets (FIG. 1, Panel e), as well as the top 20 significantly DE probe sets (FIG. 1, Panel e), showed two major clusters of R and NR for IBD overall, with 8/43 and 6/43 misclassifications, respectively. The DE probe sets from each pairwise comparison are listed in Supplementary Table 1. To identify which biological processes are associated with (non-)response to IFX treatment, we performed a GO analysis on the DE probe sets from the pairwise comparisons between R and NR in CD and IBD (Supplementary Table 2). The DE probe sets included genes that were predominantly involved in immune response, inflammatory response, signal transduction, cell adhesion, response to wounding, response to pest, pathogen or parasite, apoptosis, or chemotaxis.

### Class Prediction Analysis of R and NR:

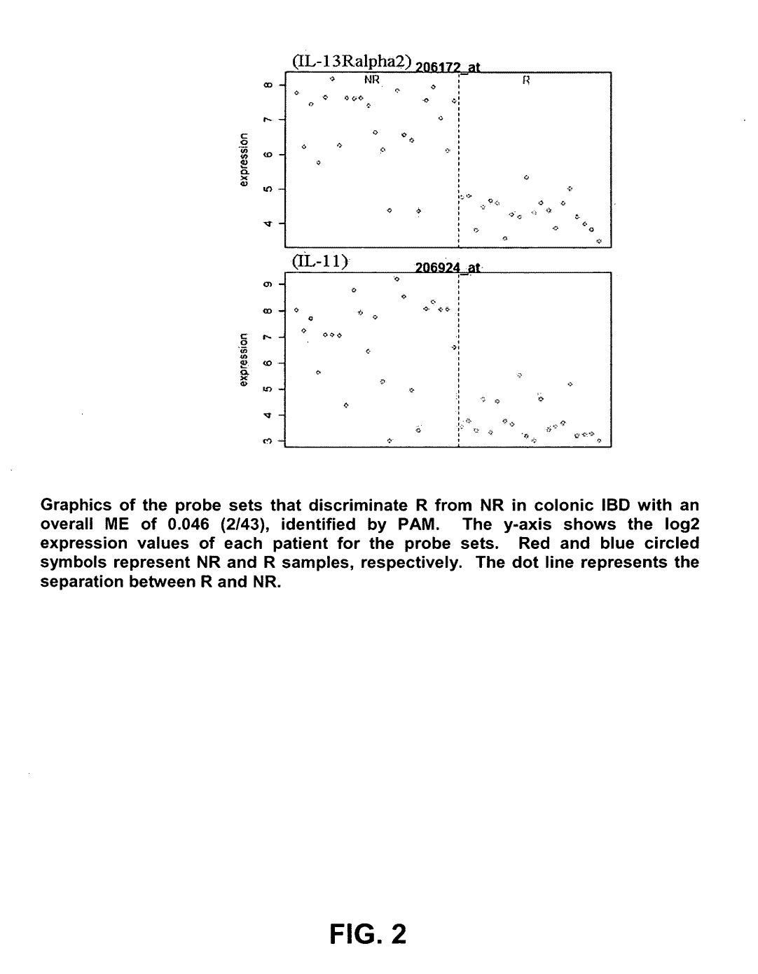

PAM was carried out on the pre-treatment expression profiles from R and NR to identify the smallest subset of probe sets that were able to accurately predict response or non-response with the lowest misclassification error (ME) rate. A subset of 37 probe sets was necessary in UC to classify samples as R or NR to achieve an overall ME rate of 0.165 (4/24) and 21 probe sets were necessary in CD to achieve a ME rate of 0 (0/15). Two probe sets, representing IL-13R(alpha)2 and IL-11, allowed nearly complete separation between R and NR in IBD overall with a ME rate of 0.046 (2/43), classifying all R (sensitivity 100%) and 21 of 23 NR (specificity 91.3%) correctly (FIG. 2). The subsets of probe sets, identified by PAM analysis in UC, CD and IBD, are listed in Supplementary Table 3.

### IL-13 R(Alpha)2:

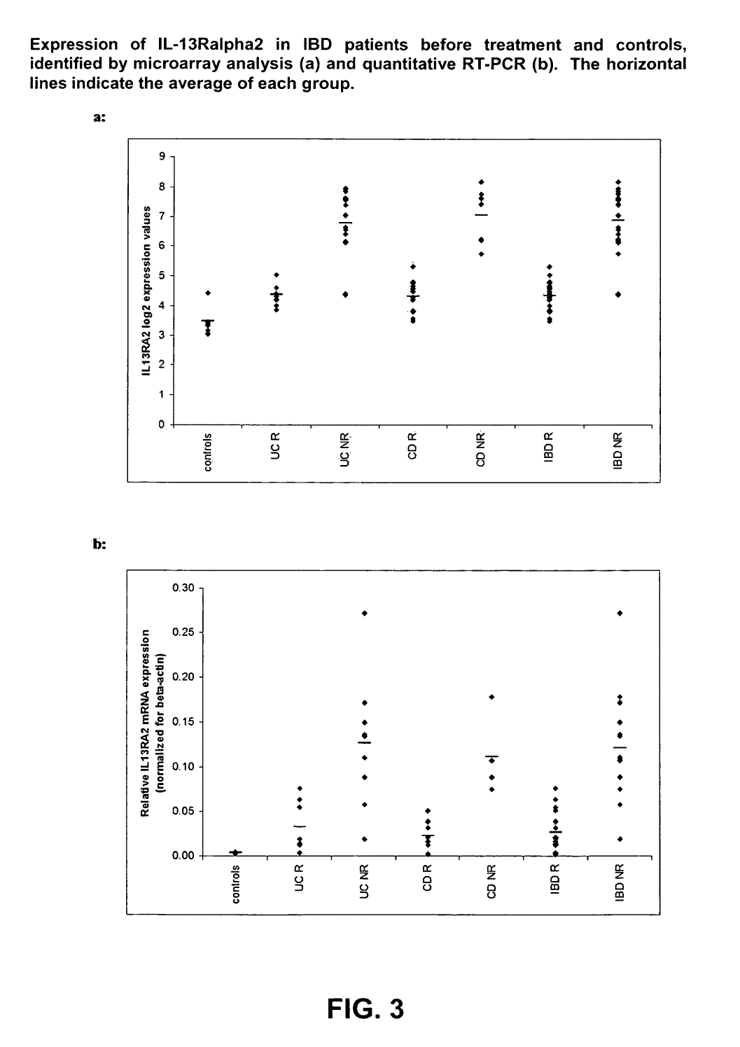

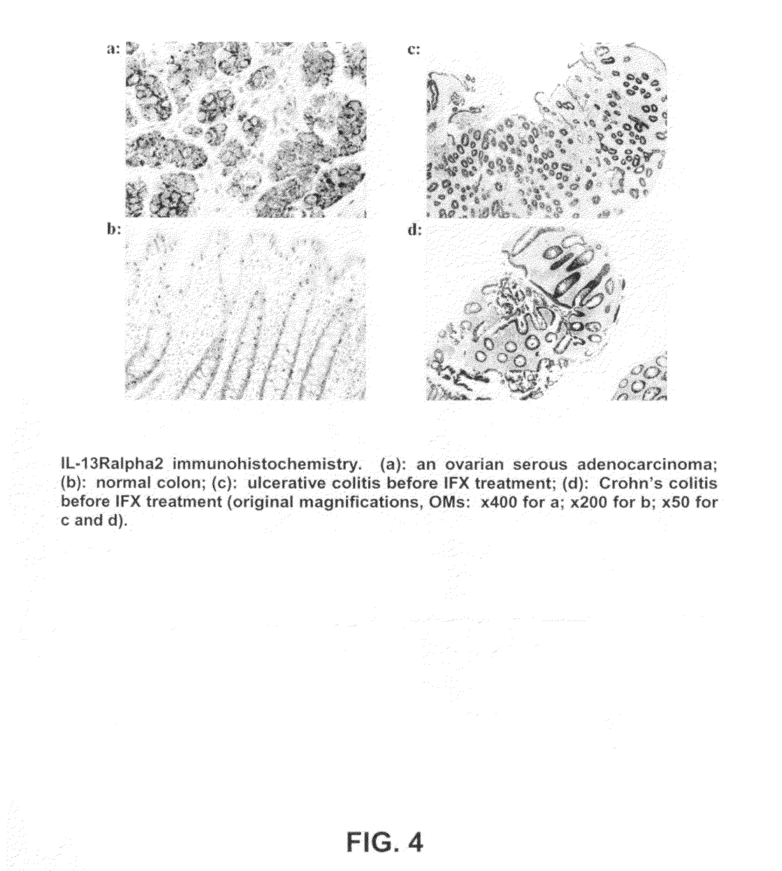

The probe set, representing IL-13R(alpha)2, was of special interest because it was present as a top-ranked probe set for both the LIMMA and PAM analyses. LIMMA analysis also showed significantly increased mRNA expression of IL-13R(alpha)2 in both untreated UC and CD compared to controls (FIG. 3, Panel a). Quantitative RT-PCR of IL-13R(alpha)2 confirmed the differential expression of IL-13R(alpha)2 between the different groups (R, NR and controls) (FIG. 3, Panel b). To determine where IL-13R(alpha)2 is expressed in the colonic mucosa, immunohistochemistry was performed. The staining for IL-13R(alpha)2 performed as expected in the positive and negative controls. In the normal colon, IL-13R(alpha)2 was located mainly in the cytoplasm of the goblet cells. Compared with normal colon, CD and UC biopsies showed an increased staining intensity in all epithelial cells. There was no restriction to goblet cells. There was also no difference in staining intensity between CD and UC (FIG. 4).

### The IL-13, IL-13R(Alpha)1, IL-13R(Alpha)2 and TGF-Beta1 Pathway:

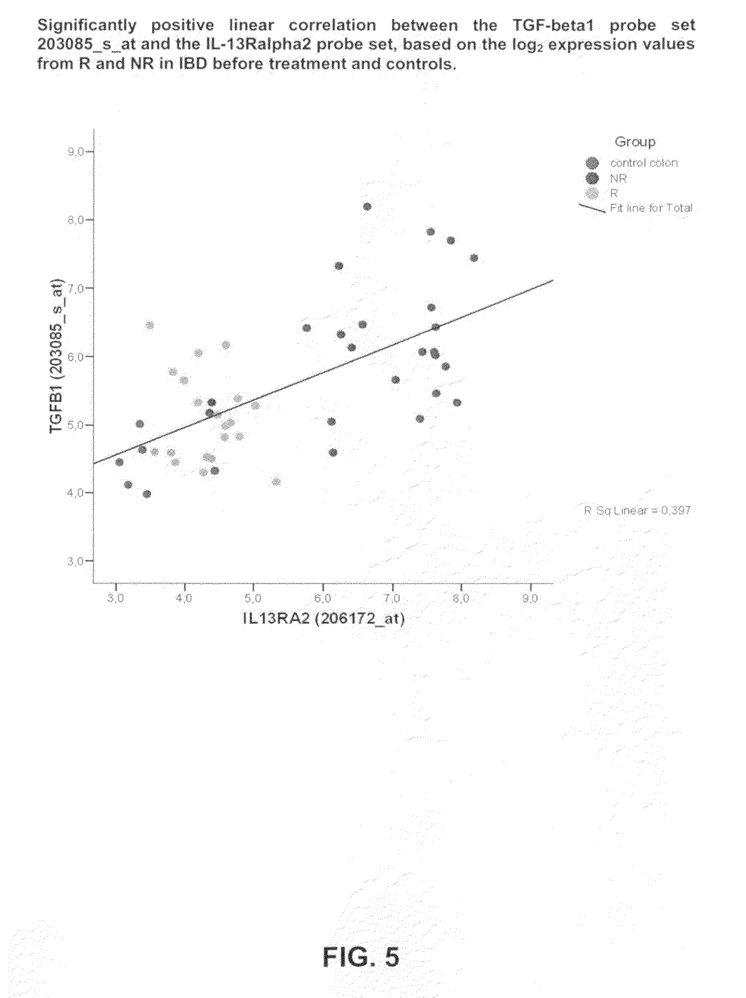

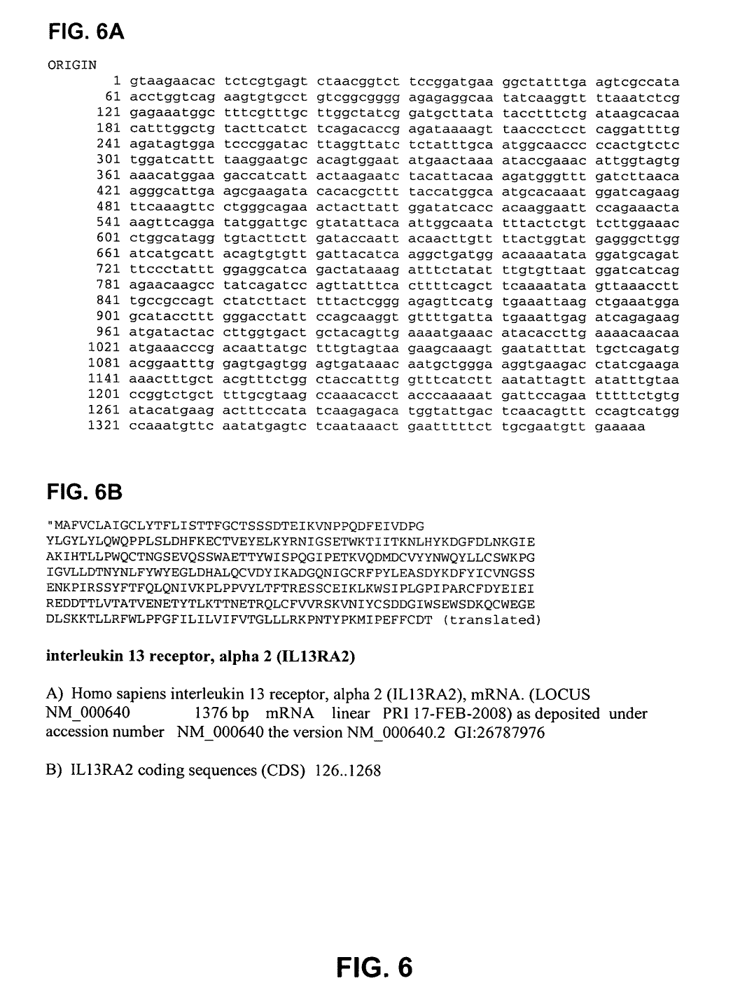

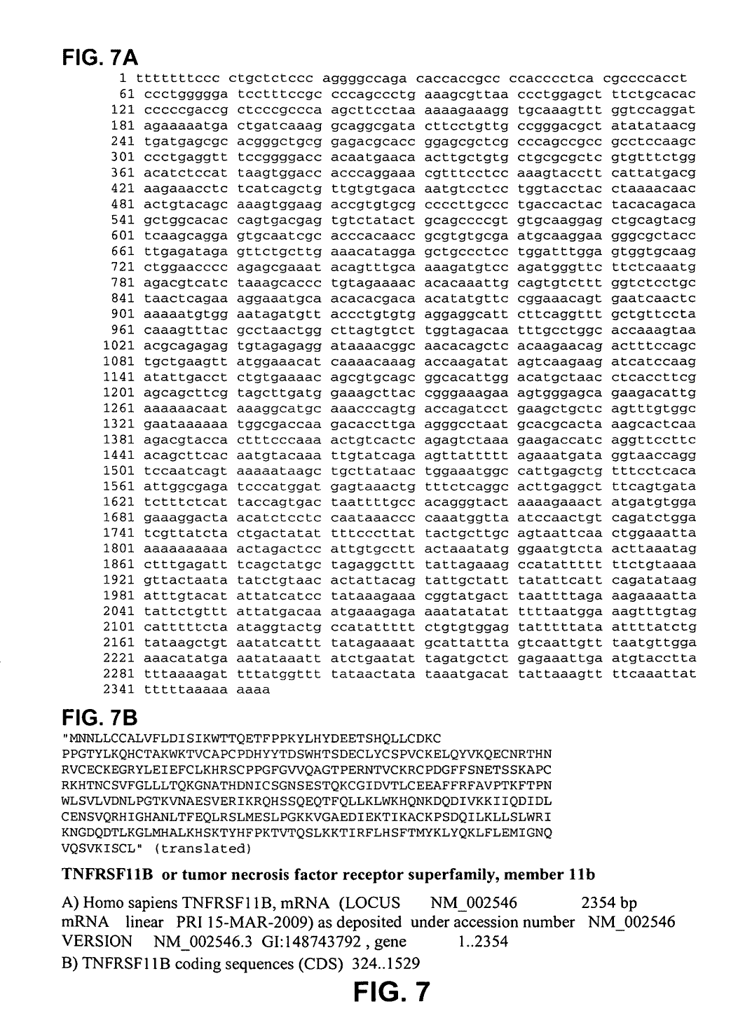

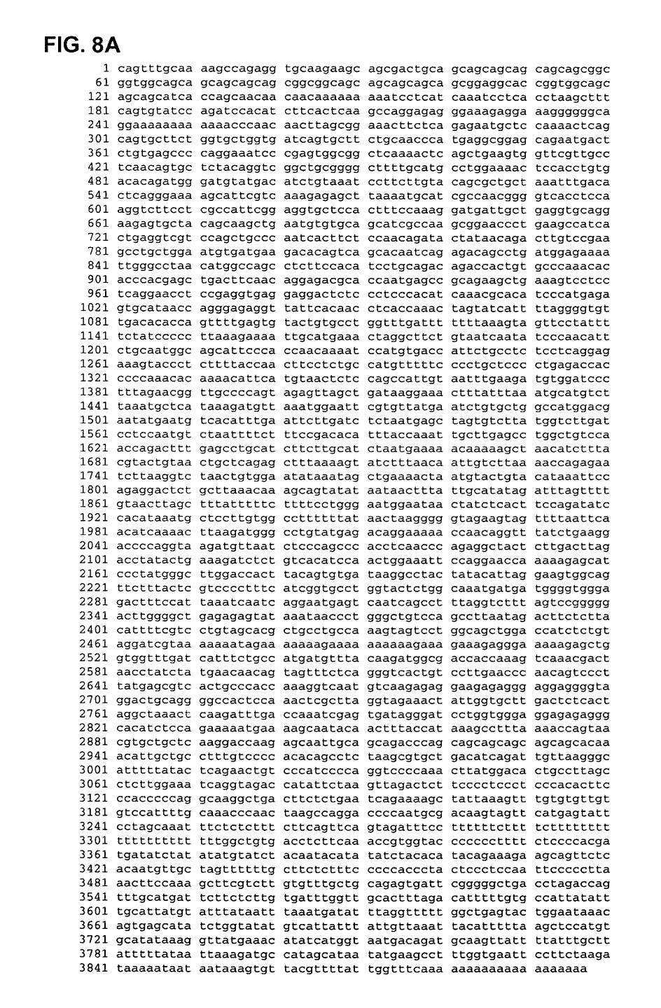

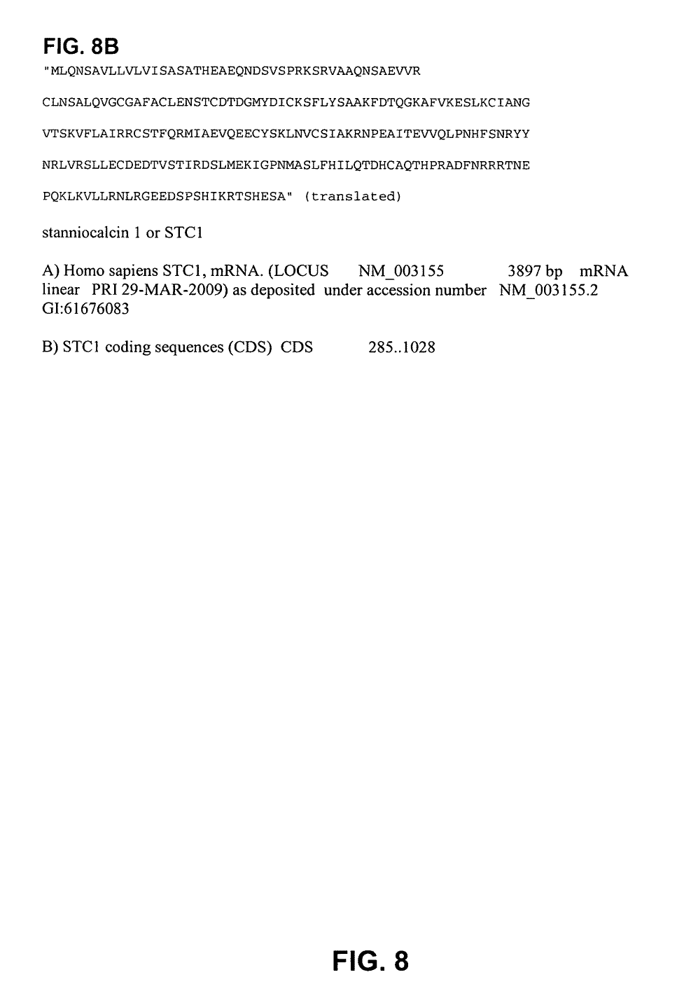

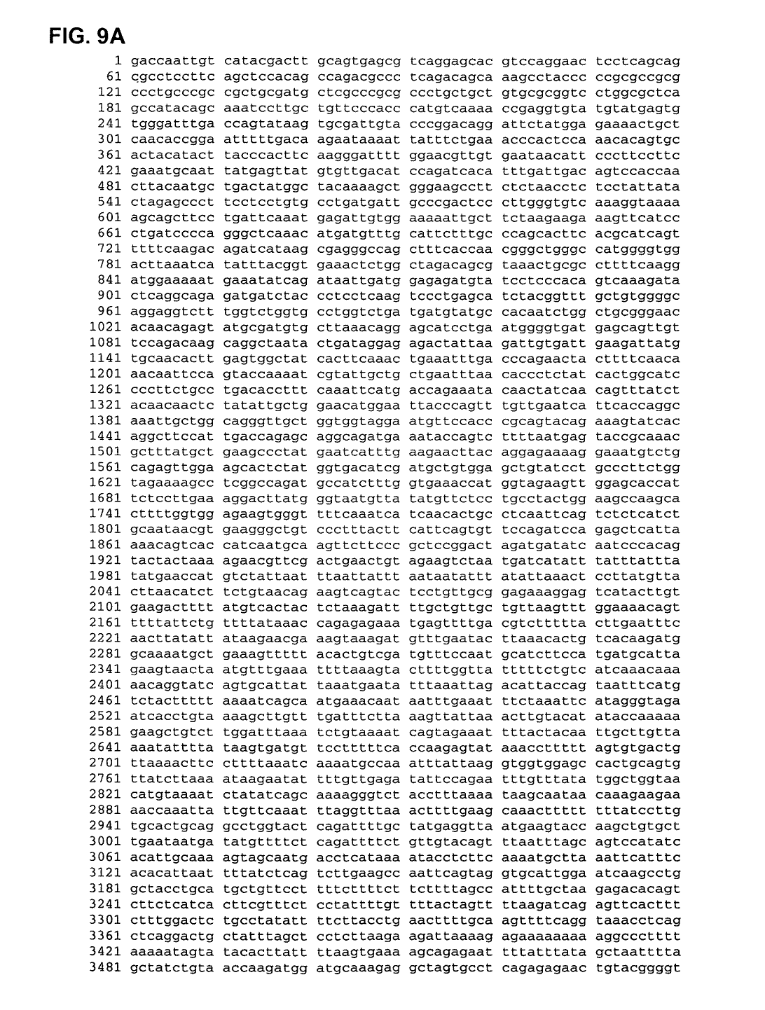

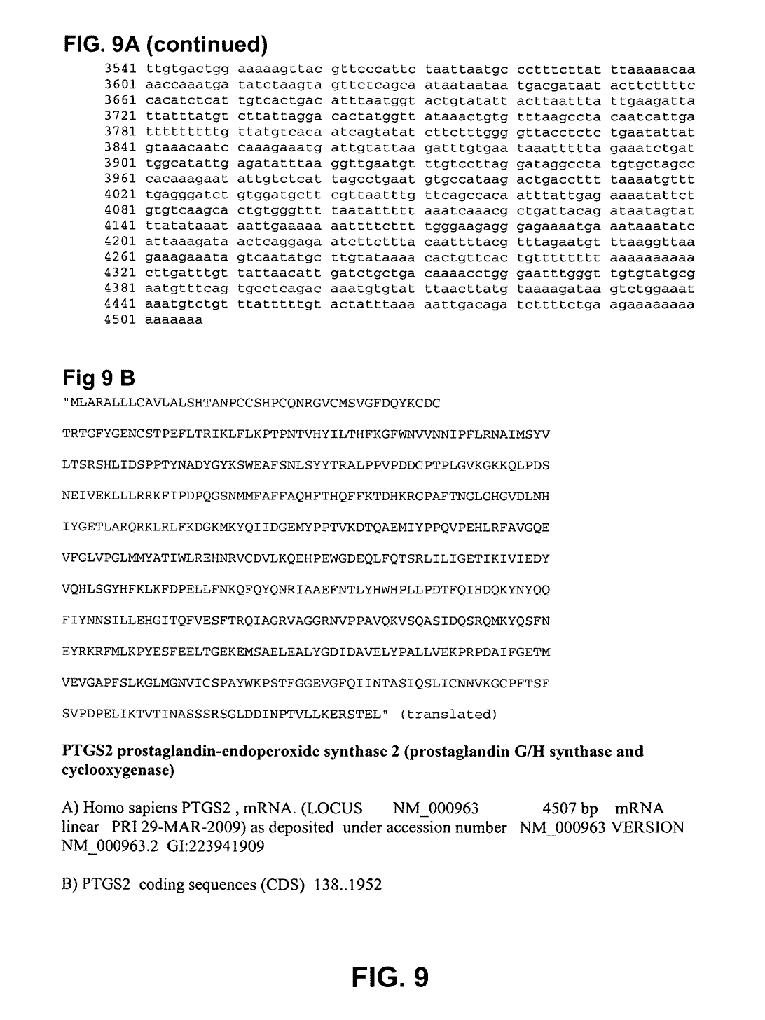

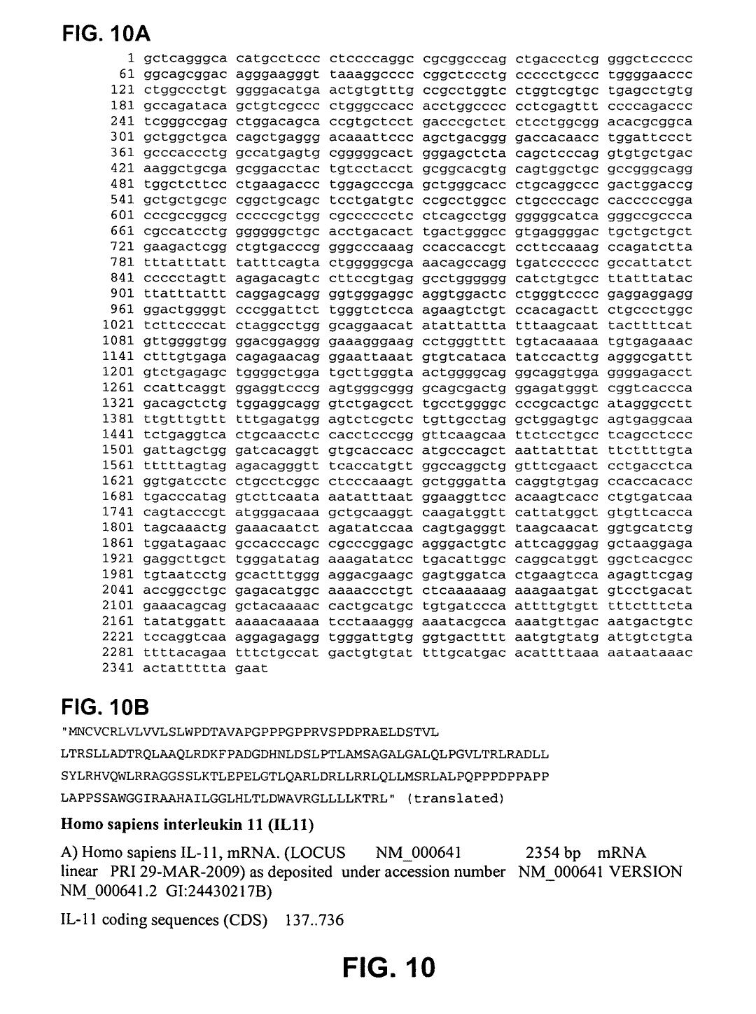

Probe sets for IL-13 and IL-13Ralpha1 were not DE between R and NR and were not up-regulated in comparison with controls. The TGF-beta1 probe set 203085_s_at and the IL-13R(alpha)2 probe set showed a significantly (P-value_{Spearman rank correlation}<0.001) positive linear correlation, based on the log₂ expression values of IBD R and IBD NR before treatment and controls (FIG. 5).

### Discussion

There is a great need to identify predictors of (non-)response to anti-TNF therapies and to biological therapies in general. We investigated whether mucosal gene signatures predictive of response to Infliximab can be identified. We studied patients with refractory IBD who had never been treated with biological therapy.

Since the reported efficacies of Infliximab in Crohn's disease and ulcerative colitis are very similar, our hypothesis was that the predictors would be the same for both diseases.

Using stringent criteria of response, namely endoscopic and histologic healing, we identified one single probe set, IL-13R(alpha)2, which was DE at LIMMA when comparing R and NR in UC. In Crohn's disease and IBD overall, many more probe sets were DE but IL-13R(alpha)2 was always one of the top-ranked probes. At PAM analysis, two probe sets, representing IL-13R(alpha)2 and IL-11, allowed separation of R and NR in IBD overall with a ME rate of 0.046 and a sensitivity of 100% and specificity of 91.3%.

In contrast with IL-11, IL-13R(alpha)2 was a top-ranked probe set in all analyses. This receptor has raised a lot of interest lately as this receptor is involved in fibrogenesis.

IL-13 is a critical regulator of a Th2 response and is the key cytokine in parasite immunity and in the generation of an allergic response. Two members of the type 5 subfamily of type I cytokine receptors can serve as receptors for IL-13. IL-13 can bind to IL-13Ralpha1 with low affinity, then recruits the IL-4Ralpha chain to form a high affinity receptor, causing downstream STAT6 activation.

Alternately, IL-13 together with TNF can induce IL-13R(alpha)2 (CD213a2) with high affinity. Interaction between IL-13 and IL-13R(alpha)2 does not activate STAT6, and originally it was believed that IL-13R(alpha)2 acts as a decoy receptor. Recently it was shown, however, that the interaction leads to activation of the TGF-Beta1 promoter.⁽¹⁹⁾

In the model of chronic TNBS colitis mimicking IBD, an initial Th1 response subsides after three weeks to be supplanted by an IL-23/IL-25 response beginning after four to five weeks. This evolution is followed by gradually increasing production of IL-17 and cytokines ordinarily seen in a Th2 response, particularly IL-13, which reaches a plateau at eight to nine weeks. IL-13 production results in the induction of IL-13R(alpha)2, inducing TGF-beta1 and the onset of fibrosis.⁽²⁰⁾

If IL-13 signaling through this receptor is blocked by administration of soluble IL-13R(alpha)2-Fc, or by administration of IL-13R(alpha)2-specific small interfering RNA, TGF-beta1 is not produced and fibrosis does not occur.

Our study shows that in UC and probably also in a subset of CD, up-regulation of IL-13R(alpha)2 is responsible for resistance to anti-TNF therapy. The effect is probably due to the induction of TGF-beta1 as we found a good correlation between expression of IL-13R(alpha)2 and TGF-beta1. Our study would suggest that IL-13R(alpha)2 expression is a good predictor of response to anti-TNF and that patients not responding to anti-TNF are candidates to receive blockade of the IL-13/IL-13R(alpha)2 pathway or of TGF-beta1. Alternatively, patients with high RT-PCR levels of IL-13R(alpha)2 might receive combined blockade of TNF and TGF-beta1 from the onset.

We identified mucosal IL-13R(alpha)2 as a predictor for response or non-response to anti-TNF therapy in IBD and, in particular, of UC.

### Example 2

### Patients

### Cohort A

Twenty-four patients with active UC, refractory to corticosteroids and/or immunosuppression, were studied in cohort A. The study was carried out at the University Hospital of Gasthuisberg in Leuven (tertiary referral center) (ClinicalTrials.gov number, NCT00639821) and all patients were followedup long term. The ethics committee of the University Hospital approved the study and all individuals gave written informed consent.

The baseline characteristics of the UC patients from cohort A are summarized in Table 1a.

TABLE 1a
Baseline characteristics of the UC patients from cohort A

| | | | Non-responders | |
|---|---|---|---|---|
| | Responders (n = 8) | | (n = 16) | |
| Male/Female (%) | 4/4 | (50/50) | 10/6 | (62.5/37.5) |
| Median (IQR) age at first IFX (years) | 28.4 | (24.3-41.8) | 45.8 | (36.5-62.3) |
| Median (IQR) weight at first IFX (kg) | 72 | (57.8-78.5) | 73.3 | (68.5-80.3) |
| Median (IQR) duration of disease prior to first IFX (years) | 10.3 | (4.1-17.3) | 7.3 | (2.6-13.3) |
| Median (IQR) C-reactive protein at first IFX (mg/dL) | 1.65 | (1-9.6) | 6.5 | (2.9-19.1) |
| Concomitant medication at first IFX (%) | | | | |
| 5-Aminosalicylates | 5 | (62.5) | 13 | (81.3) |
| Corticosteroids | 2 | (25) | 5 | (31.3) |
| Azathioprine/6-Mercaptopurine | 7 | (87.5) | 8 | (50) |
| Corticosteroids + Immunosuppressants | 2 | (25) | 1 | (6.3) |
| Active smoking at first IFX (%) | 1 | (12.5) | 1 | (6.3) |

IQR, interquatile range;
IFX, infliximab

In the study, consecutive patients with active UC who consented with the study were included. The patients had a total Mayo score between 6 and 12 and an endoscopic subscore of at least 2 (P. Rutgeerts, W. J. Sandborn, and B. G. Feagan et al., Infliximab for induction and maintenance therapy for ulcerative colitis, N. Engl. J. Med. 2005; 353(23):2462-76). The UC patients underwent colonoscopy with biopsies from diseased rectum within a week prior to the first intravenous infusion of 5 mg Infliximab per kg body weight. The patients underwent a second flex sigmoidoscopy with rectal biopsies four weeks after the first Infliximab infusion in case of a single infusion and at six weeks if they received a loading dose of Infliximab at weeks 0, 2 and 6. The endoscopist was not blinded to treatment. Half of the biopsies were immediately snap-frozen in liquid nitrogen and stored at −80° C. until RNA isolation. The residual biopsies were fixed in Carnoy's fixative for up to five hours and then dehydrated, cleared and paraffin-embedded for histologic examination. The features of chronic intestinal inflammation were scored in haematoxylin-eosin stained slides from the paraffin blocks of each patient using a previously reported scoring system for UC (K. Geboes, R. Riddell, and A. Ost et al., A reproducible grading scale for histological assessment of inflammation in ulcerative colitis, Gut 2000; 47(3):404-9). The pathologists who scored the biopsies (KG and GDH) were blinded to treatment.

The response to Infliximab was assessed at four to six weeks after the first Infliximab treatment and defined as a complete mucosal healing with a Mayo endoscopic subscore of 0 or 1 (P. Rutgeerts, W. J. Sandborn, and B. G. Feagan et al., Infliximab for induction and maintenance therapy for ulcerative colitis, N. Engl. J. Med. 2005; 353(23):2462-76) and a grade 0 or 1 on the histological score for UC (K. Geboes, R. Riddell, and A. Ost et al., A reproducible grading scale for histological assessment of inflammation in ulcerative colitis, Gut 2000; 47(3):404-9). Patients who did not achieve this healing were considered non-responders although some of them presented endoscopic and/or histologic improvement.

For validation of the microarray results with quantitative RT-PCR (qPCR), colonic biopsies were obtained from six control subjects who underwent colonoscopy for screening for polyps. These patients gave informed consent for the study.

### Cohort B

Endoscopies were carried out and biopsies were collected during the ACT1 trial (P. Rutgeerts, W. J. Sandborn, and B. G. Feagan et al., Infliximab for induction and maintenance therapy for ulcerative colitis, N. Engl. J. Med. 2005; 353(23):2462-76), a placebo-controlled trial of Infliximab therapy in refractory UC (ClinicalTrials.gov number NCT00036439), at protocol-specified time points from a subset of randomized patients. Endoscopists and pathologists in this study were blinded to therapy. The institutional review board or ethics committee at each site approved the ACT1 protocol and all patients provided informed consent.

For the current study, 23 pre-treatment colonic mucosal biopsies from 22 active UC patients (two colonic mucosal biopsies were obtained within two weeks from the same patient) who received their first infusion of Infliximab 5 or 10 mg/kg for refractory ulcerative colitis were analyzed in cohort B. The biopsies were collected 15 to 20 centimeters distal from the anal verge. Table 1b summarized the baseline characteristics of the UC patients from cohort B.

TABLE 1b
Baseline characteristics of the UC patients from cohort B

| | Responders | | Non-responders | |
|---|---|---|---|---|
| | (n = 12) | | (n = 10) | |
| Male/Female (%) | 6/6 | (50/50) | 6/4 | (60/40) |
| Median (IQR) age at first IFX (years) | 39 | (29-70) | 51.5 | (24-68) |
| Median (IQR) weight at first IFX (kg) | 75 | (63-159) | 69 | (46-102) |
| Median (IQR) duration of disease prior to first IFX (years) | 5.9 | (1.6-42.1) | 5.7 | (2.9-26.8) |
| Median (IQR) C-reactive protein at first IFX (mg/dL) | 0.7 | (0.2-2.9) | 1.35 | (0.2-6.8) |
| Concomitant medication at first IFX (%) | | | | |
| 5-Aminosalicylates | 1 | (8.3) | 1 | (10) |
| Corticosteroids | 8 | (66.7) | 6 | (60) |
| Azathioprine/6-Mercaptopurine | 2 | (16.7) | 3 | (30) |
| Corticosteroids + Immunosuppressants | 0 | (0) | 0 | (0) |
| Active smoking at first IFX (%) | 1 | (8.3) | 0 | (0) |

IQR, interquatile range;
IFX, infliximab

The response to Infliximab was assessed at eight weeks after their first Infliximab treatment and the definition of response was identical to cohort A.

### RNA Isolation:

For cohort A, total RNA was extracted from the biopsy specimens using the RNEASY® Mini Kit (Qiagen, Benelux B.V.), according to the manufacturer's instructions. The integrity and quantity of total RNA were assessed with a 2100 BIOANALYZER® (Agilent, Waldbronn, Germany) and NANODROP® ND-1000 spectrophotometer (Nanodrop Technologies). The extracted RNA was used for microarray analysis and in some cases for qPCR analysis.

For cohort B, total RNA was isolated from the biopsies with RNEASY® mini-kit according to the manufacturer's instructions (Qiagen, Valencia, Calif.). RNA quality and quantity were analyzed with a 2100 BIOANALYZER® (Agilent Technologies Inc., Palo Alto, Calif.).

### Oligonucleotide Array Hybridization:

For samples from both cohorts, all steps were performed according to AFFYMETRIX® expression analysis technical manual 701021Rev.5 (Affymetrix, Santa Clara, Calif., USA). Briefly, total RNA (2 μg) was reverse-transcribed into cDNA using the SUPERSCRIPT® Choice System (Invitrogen, Carlsbad, Calif., USA). cDNA was in vitro transcribed to cRNA and biotin labeled (Affymetrix). Biotinylated cRNA was purified and fragmented. The quality of labeled and fragmented cRNA, respectively, was assessed with the AGILENT® 2100 BIOANALYZER®. Fragmented cRNA (15 μg) was hybridized overnight to the Human Genome U133 Plus 2.0 Array (Affymetrix), which comprised of 54675 probe sets. The arrays were washed, stained with streptavidin-phycoerytrin and scanned on the AFFYMETRIX® 3000 GeneScanner. The resulting image files (.dat files) were analyzed using AFFYMETRIX® GCOS software, and intensity values for each probe cell (.cel file) were calculated. Quality evaluations of the microarrays were as expected.

### Data Analysis:

A glossary of terms used in the analysis is provided in Supplementary Appendix 1.

The microarray data were analyzed using Bioconductor tools (R. C. Gentleman, V. J. Carey, D. M. Bates et al., Bioconductor: open software development for computational biology and bioinformatics, Genome Biol. 2004; 5(10):R80) in R (version 2.7.2, http://r-project.org/). The robust multichip average (RMA) method (R. A. Irizarry, B. Hobbs, and F. Collin et al., Exploration, normalization, and summaries of high density oligonucleotide array probe level data, Biostatistics 2003; 4(2):249-64) was performed on the AFFYMETRIX® raw data (.cel files) from both cohorts to obtain an expression value for each probe set. Because the severe correction for 54675 comparisons may have caused false negatives, we next performed the analysis with probe sets that hybridized above background levels to the patient samples. The probe sets with low overall intensity and variability that are unlikely to carry information about the phenotypes under investigation were removed. A non-specific filtering was applied on the log2 RMA normalized data from the pre-treatment UC samples from both cohorts. Only probe sets with an intensity>log2(100) in at least 10% of the samples and an interquartile range (IQR) of log2 intensities across the samples>0.5 were included, leaving 9183 probe sets for further data analysis (Supplementary Table 1). Probe set annotations were obtained through the AFFYMETRIX® NetAffx website (http://affymetrix.com/analysis/index.affx) or the UCSC Genome Browser website (http://genome.ucsc.edu/). Linear models for microarray data (LIMMA) (G. K. Smyth, Linear models and empirical bayes methods for assessing differential expression in microarray experiments, Stat. Appl. Genet. Mol. Biol. 2004; 3:Article 3) and prediction analysis of microarrays (PAM) (R. Tibshirani, T. Hastie, B. Narasimhan et al., Diagnosis of multiple cancer types by shrunken centroids of gene expression, Proc. Natl. Acad. Sci. U.S.A. 2002; 99(10):6567-72) were used for supervised data analyses. For comparative analysis, LIMMA was used to identify probe sets that are different between responders and non-responders, based on moderated t-statistics. To correct for multiple testing, the false discovery rate (FDR) was estimated from p-values derived from the moderated t-statistics using the method of Benjamini and Hochberg.⁽¹¹⁾ A greater than two-fold change combined with a FDR<0.05 were considered statistically significant. For class prediction, PAM with leave-one-out cross-validation was carried out on the top twenty and the top five most significantly different known genes, identified by LIMMA analysis between responders and non-responders, to see if these subsets accurately predict response or non-response to Infliximab and to identify the lowest misclassification error rate based on these subsets. Unsupervised average-linkage hierarchical clustering, using Euclidian distance as metric, was applied to data obtained from the data analysis to visualize gene/sample relationship. The results of the clustering were visualized as a two-dimensional heatmap with two dendograms, one indicating the similarity between patients and the other indicating the similarity between genes. The Bio Functional Analysis tool in the Ingenuity Pathway Analysis program (INGENUITY® Systems, on the world-wide web at ingenuity.com) was used to identify biological functions and/or diseases that were most significant to the dataset of significant probe sets that were identified by LIMMA analysis between responders and non-responders. The genes represented by the significant probe sets that were associated with biological functions and/or diseases in the Ingenuity knowledge base were considered for the analysis. Fischer's exact test was used to calculate a p-value determining the probability that each biological function and/or disease assigned to that data set is due to chance alone. For multiple testing correction, the p-values were adjusted with the Benjamini and Hochberg (B-H) method.

### Quantitative RT-PCR:

To validate the microarray data, qPCR was performed for osteoprotegerin (TNFRSF11B), stanniocalcin-1 (STC1), prostaglandin-endoperoxide synthase 2 (PTGS2), interleukin-13 receptor alpha 2 (IL-13R(alpha)2), interleukin-11 (IL-11) and beta-actin. Beta-actin was used as the endogenous reference gene. Total RNA from samples of cohort A was used. cDNA was synthesized from 0.5 μg of total RNA using the REVERTAID™ H Minus First Strand cDNA synthesis kit (Fermentas, St. Leon-Rot, Germany), following the manufacturer's protocol. Primers and dual-labeled probes were designed using OligoAnalyzer 3.0 software (http://biotools.idtdna.com/analyzer/) and synthesized by Sigma-Genosys Ltd. (Haverhill, UK). The oligonucleotide sequences are available upon request. Duplex qPCR was performed in a final reaction volume of 25 μl on a ROTOR-GENE® 3000 instrument (Corbett Research Pty Ltd., Mortlake, Australia), using QUANTITECT® Multiplex PCR NoROX Kit (Qiagen, Venlo, NL), according to the manufacturer's instructions. Cycle threshold values were determined by ROTOR-GENE® 6.0.16 software. All samples were amplified in duplicate reactions. The relative expression of target mRNA levels were calculated as a ratio relative to the beta-actin reference mRNA (M. W. Pfaffl, A new mathematical model for relative quantification in real-time RT-PCR, Nucleic Acids Res. 2001; 29(9):e45). Results were analyzed using the Mann-Whitney U-test using SPSS 16.0 software (SPSS, Chicago, Ill.) and a P-value of <0.05 was considered significant.

### Results

### Response to Infliximab:

Two independent UC cohorts (cohort A and cohort B) were studied to identify mucosal gene signatures predictive of response to Infliximab in UC. The response to Infliximab was defined as complete endoscopic and histologic healing. Of the 24 UC patients in cohort A, eight patients were responders and sixteen patients were non-responders, while cohort B had twelve responders and ten non-responders.

### Comparative Analysis Between Responders and Non-Responders:

For predicting response to Infliximab treatment based on gene profiles, pre-treatment expression profiles were compared for responders and non-responders in each cohort and both cohorts combined, using LIMMA.

When all probe sets on the microarray (54675 probe sets) were included in the LIMMA analysis, the stringent correction for multiple testing resulted in only one significant probe set between responders and non-responders for UC in cohort A, namely IL-13R(alpha)2. Therefore, a non-specific filtering was first applied on the normalized data from the pre-treatment UC samples from both cohorts to eliminate non-relevant probe sets, leaving 9183 probe sets for further comparative analyses (Supplementary Table 1).

In cohort A, LIMMA analysis identified a total of 179 probe sets that were significantly decreased in responders compared with non-responders (Table 2 and Supplementary Table 2). In cohort B, a total of 361 probe sets were significantly different in responders compared to non-responders, with 38 probe sets showing an increased signal and 323 probe sets a decreased signal in responders compared to non-responders (Table 2 and Supplementary Table 2). For the two cohorts combined (cohort A and cohort B), a total of 212 significant probe sets were identified by LIMMA analysis, with five probe sets showing an increased signal and 207 probe sets a decreased signal in responders compared to non-responders (Table 2 and Supplementary Table 2).

TABLE 2
Summary of the results from LIMMA analyses between the
pre-treatment expression profiles of responders and
non-responders in cohort A, cohort B and both cohorts combined

| | US cohort A | UC cohort B | UC cohort A and B |
|---|---|---|---|
| | R (n = 8)/ | R (n = 12)/ | R (n = 20)/ |
| Comparative analysis | NR (n = 16) | NR (n = 10) | NR (n = 26) |
| Increased probe sets | 0 | 38 | 5 |
| in R | | | |
| Decreased probe sets | 179 | 323 | 207 |
| in R | | | |
| Total | 179 | 361 | 212 |

NR, non-responders;
R, responders

There was an overlap of 74 significant probe sets, representing 53 different known genes, between the LIMMA analyses in cohort A and in cohort B, and these common probe sets were all down-regulated in responders as compared to non-responders (Table 3).

TABLE 3
Fold change of the 74 common significant probe sets between
LIMMA analysis in cohort A and LIMMA analysis in cohort B

| | | Cohort A | Cohort B | | | Cohort A | Cohort B |
|---|---|---|---|---|---|---|---|
| Probe Set ID* | Gene Symbol | FC (R/NR) | FC (R/NR) | Probe Set ID | Gene Symbol | FC (R/NR) | FC (R/NR) |
| 202422_s_at | ACSL4 | 0.30 | 0.33 | 229947_at | PI15 | 0.14 | 0.09 |
| 226517_at | BCAT1 | 0.39 | 0.47 | 220014_at | PRE16 | 0.39 | 0.42 |
| 204103_at | CCL4 | 0.38 | 0.39 | 1554997_a_at | PTGS2 | 0.21 | 0.12 |
| 219947_at | CLEC4A | 0.49 | 0.44 | 204748_at | PTGS2 | 0.22 | 0.13 |
| 231766_s_at | COL12A1 | 0.28 | 0.39 | 223809_at | RG S18 | 0.46 | 0.41 |
| 205159_at | CSF2RD | 0.42 | 0.42 | 209071_s_at | RG S5 | 0.46 | 0.49 |
| 214974_x_at | CXCL5 | 0.23 | 0.10 | 203535_at | S100A9 | 0.39 | 0.20 |
| 206336 at | CXCL6 | 0.22 | 0.25 | 220330 s at | SAMSN1 | 0.35 | 0.43 |
| 207610_s_at | EMR2 | 0.40 | 0.37 | 1555638_a_at | SAMSN1 | 0.38 | 0.39 |
| 217967_s_at | FAM129A | 0.36 | 0.38 | 206211_at | SELE | 0.27 | 0.17 |
| 217966_s_at | FAM129A | 0.47 | 0.44 | 202627_s_at | SERPINE 1 | 0.40 | 0.38 |
| 1554899_s_at | FCER1G | 0.42 | 0.39 | 202498_s_at | SL C2A3 | 0.41 | 0.31 |
| 203561_at | FCGR2A | 0.39 | 0.33 | 218404_at | SNX10 | 0.49 | 0.48 |
| 1554741_s_at | FGF7 | 0.33 | 0.38 | 227697_at | SOCS3 | 0.41 | 0.29 |
| 229435_at | GLIS3 | 0.41 | 0.43 | 201858_s_at | SRGN | 0.40 | 0.36 |
| 211959_at | IGFBP5 | 0.28 | 0.43 | 204597_x_at | STC1 | 0.26 | 0.24 |
| 203424_s_at | IGFBP5 | 0.45 | 0.34 | 230746_s_at | STC1 | 0.19 | 0.21 |
| 206420_at | IGSF6 | 0.48 | 0.35 | 204595_s_at | STC1 | 0.35 | 0.28 |
| 206924_at | IL11 | 0.13 | 0.17 | 229723_at | TAGAP | 0.30 | 0.24 |
| 206172_at | IL13RA2 | 0.22 | 0.20 | 210664_s_at | TFPI | 0.50 | 0.45 |
| 205207_at | IL6 | 0.22 | 0.17 | 209278_s_at | TFPI2 | 0.16 | 0.15 |
| 205798_at | IL7R | 0.45 | 0.49 | 210176_at | TLR1 | 0.41 | 0.32 |
| 210511_s_at | INHBA | 0.23 | 0.22 | 201645_at | TNC | 0.22 | 0.32 |
| 226001_at | KLHL5 | 0.39 | 0.49 | 204933_s_at | TNFRSF11B | 0.32 | 0.23 |
| 205269_at | LCP2 | 0.40 | 0.42 | 204932_at | TNFRSF11B | 0.28 | 0.24 |
| 229937_x_at | LILRB1 | 0.42 | 0.44 | 231227_at | WNT5A | 0.25 | 0.39 |
| 210146_x_at | LILRB2 | 0.33 | 0.26 | 205990_s_at | WNT5A | 0.28 | 0.32 |
| 206953_s_at | LPHN2 | 0.42 | 0.45 | 213425_at | WNT5A | 0.31 | 0.32 |
| 206584_at | LY96 | 0.38 | 0.46 | 232297_at | N/A | 0.44 | 0.49 |
| 220122_at | MCTP1 | 0.44 | 0.47 | 227140_at | N/A | 0.16 | 0.17 |
| 203434_s_at | MME | 0.33 | 0.39 | 209960_at | N/A | 0.42 | 0.43 |
| 205828_at | MMP3 | 0.25 | 0.30 | 242388_x_at | N/A | 0.42 | 0.37 |
| 224940_s_at | PAPPA | 0.39 | 0.39 | 226802_at | N/A | 0.33 | 0.28 |
| 228128_x_at | PAPPA | 0.35 | 0.38 | 226218_at | N/A | 0.41 | 0.46 |
| 224941_at | PAPPA | 0.39 | 0.43 | 226847_at | N/A | 0.36 | 0.37 |
| 203708_at | PDE4B | 0.41 | 0.31 | 226237_at | N/A | 0.36 | 0.32 |
| 211302_s_at | PDE4B | 0.44 | 0.29 | 209683_at | N/A | 0.44 | 0.47 |

FC, fold change; N/A, not available; NR, non responders; NR, non responders
*Full annotation for each probe set is given in Supplementary table 1

Ten overlapping genes came up in the top twenty significantly differentially expressed known genes for cohort A and three overlapping genes belonged to the top twenty genes of cohort B. The top ten most significant biological functions that were associated with the list of the common probe sets.

To identify which biological processes and/or diseases are associated with (non-)response to Infliximab treatment, a Bio Functional Analysis was performed on the significant probe sets from each LIMMA analysis (Supplementary Table 3). The Bio Functional analyses showed a common predominance of the biological functions: immune response, cellular movement, cellular growth and proliferation, hematological system development and function, cell-to-cell signaling and interaction, cell death and tissue morphology/development.

### Class Prediction Analysis of Responders and Non-Responders:

PAM analysis was carried out on the top twenty and top five most significantly different known genes that were identified by LIMMA analysis between responders and non-responders in cohort A, cohort B and both cohorts combined.

In cohort A, PAM analysis of the top twenty and top five genes allowed classification of samples as responder and non-responder with an overall accuracy of 91.7% (22/24) and 83.3% (20/24), respectively. The top twenty and top five gene classifier of cohort A were used to predict the (non-)response of the samples from cohort B. Both cohort A classifiers predicted 3/12 responders (25% sensitivity) and 10/10 non-responders (100% specificity) in cohort B correctly. Hierarchical clustering of the log2 expression values from the top twenty and top five genes in cohort A resulted in two major clusters of responders versus non-responders, with two non-responders misclassified in the cluster of responders.

In cohort B, PAM analysis of the top twenty and top five genes that were identified by LIMMA analysis in cohort B revealed an overall accuracy of 86.4% (19/22) and 90.9% (20/22), respectively. The top twenty and top five gene classifiers of cohort B predicted the samples from cohort A with an overall accuracy of 66.7% and 70.8%, respectively.

For the two cohorts combined, PAM analysis of the top twenty and top five genes from the LIMMA analysis for both cohorts combined allowed classification of responders and non-responders with an overall accuracy of 84.8% (39/46) and 89.1% (41/46), respectively. For the two cohorts combined, PAM analysis showed that a predictive signature of twenty gene probes was not better than a panel of five gene probes to differentiate responders from non-responders.

The subset of the top twenty and top five gene classifiers used for the PAM analyses in cohort A, cohort B and both cohorts combined are listed in Supplementary Table 4. Table 4 summarizes the results (overall accuracy, sensitivity and specificity) of the PAM analyses.

Table 4: Summary of the results from PAM analyses in cohort A, cohort B and both cohorts combined

PAM analysis

| UC cohort A (R = 8, NR = 16) | Sensitivity | | Specificity | | Overall accuracy | |
|---|---|---|---|---|---|---|
| Top 20 genes | 100% | (8/8) | 87.5% | (14/16) | 91.7% | (22/24) |
| Top 5 genes | 75% | (6/8) | 87.5% | (14/16) | 83.3% | (20/24) |
| The top-ranked gene classifiers of | | | | | | |
| cohort A were used to classify | Sensitivity in | | Specificity in | | Overall accuracy | |
| samples in cohort B (R = 12, NR = 10) | cohort B | | cohort B | | in cohort B | |
| Cohort A top 20 genes | 25% | (3/12) | 100% | (10/10) | 59.1% | (13/22) |
| Cohort A top 5 genes | 25% | (3/12) | 100% | (10/10) | 59.1% | (13/22) |
| UC cohort B (R = 12, NR = 10) | Sensitivity | | Specificity | | Overall accuracy | |
| Top 20 genes | 91.7% | (11/12) | 80% | (8/10) | 86.4% | (19/22) |
| Top 5 genes | 91.7% | (11/12) | 90% | (9/10) | 90.9% | (20/22) |
| The top-ranked gene classifiers of | | | | | | |
| cohort A were used to classify | Sensitivity in | | Specificity in | | Overall accuracy | |
| samples in cohort B (R = 12, NR = 10) | cohort A | | cohort A | | in cohort A | |
| Cohort B top 20 genes | 87.5% | (7/8) | 56.3% | (9/16) | 66.7% | (16/24) |
| Cohort B top 5 genes | 87.5% | (7/8) | 62.5% | (10/16) | 70.8% | (17/24) |
| UC cohort A and B (R = 20, NR = 26) | Sensitivity | | Specificity | | Overall accuracy | |
| Top 20 genes | 95% | (19/20) | 76.9% | (20/26) | 84.8% | (39/46) |
| Top 5 genes | 95% | (19/20) | 84.6% | (22/26) | 89.1% | (41/46) |

NR, non-responders;
R, responders

Validation of the Microarray Data by qPCR:

To confirm the microarray data, qPCR was performed for the top five significant known genes of the LIMMA analysis of the two cohorts combined (TNFRSF11B, STC1, PTGS2, IL-13R(alpha)2 and IL-11). LIMMA analysis in cohort A also showed significantly increased mRNA expression of these genes in untreated UC when compared to controls. Quantitative RT-PCR confirmed the differential expression of these genes between the responders, non-responders and controls in cohort A.

### Discussion

There is a great need for defining molecular mechanisms that underlie response or non-response to anti-TNFα therapy in UC. In this study, we investigated endoscopic mucosal biopsy derived mRNA expression to identify a “gene expression fingerprint” distinguishing primary non-responders from responders and to identify gene panels predictive of response to Infliximab, a chimeric IgG1 monoclonal antibody to TNFα. Two independent cohorts of patients with refractory UC who received a first treatment with Infliximab were studied. The first cohort included 24 consecutive patients treated at the University Hospital in Leuven, Belgium, and the second cohort consisted of 22 patients who were treated in the ACT1 clinical trial in UC. Although in the ACT1 trial clinical response was used as an endpoint for the study, we chose to use a more stringent criteria for response to Infliximab in this study, i.e., complete endoscopic and histologic healing at the preset time point. We classified patients as responders if they achieved complete healing, both endoscopically and histologically. All other patients were considered non-responders although some of them showed clinical improvement or showed mucosal healing at a later time point. We used mucosal healing as an endpoint as it is less open to bias than a symptom score, and there is little chance that complete mucosal healing would occur only by spontaneous disease evolution. Moreover, in this manner, we were able to correlate endoscopic activity with histologic activity. Combined endoscopic and histologic healing minimizes the placebo effect on the results, with the endoscopic results providing a distinct signature based upon the biological activity of the anti-TNF agent. In cohort A, endoscopic evaluation was performed in an open fashion by an endoscopist who was informed about the treatment but histology was blinded. In cohort B, both endoscopic and histologic assessment were blinded as the patients in this cohort took part in the ACT1 placebo-controlled clinical trial with Infliximab in UC (P. Rutgeerts, W. J. Sandborn, and B. G. Feagan et al., Infliximab for induction and maintenance therapy for ulcerative colitis, N. Engl. J. Med. 2005; 353(23):2462-76).

Microarray analysis of pre-treatment mucosal biopsies for both cohorts A and B yielded 179 and 323 probe sets, respectively, that were significantly down-regulated in responders versus non-responders. Although the differentially expressed probe sets were not the same for the two cohorts, a comparative analysis of cohort A and B showed an overlap for 74 probe sets differentially expressed, representing 53 different known genes. The top biological functions that were over-represented within the lists of significant probe sets were cellular movement, hematological system development and function, immune response and cell death. It is not surprising that there is no perfect overlap for the signatures found in both cohorts. This is likely due to differences in patient populations, different environmental background and concomitant therapies.

For each cohort, the top significant gene probes were used for predicting response to Infliximab in UC and the overall accuracy with the sensitivity and specificity were calculated. The overall accuracy of the top twenty and top five genes for cohort A were 92% and 83%, respectively. For cohort B, overall accuracy was 86% and 91% for the top twenty and top five genes, respectively. When using cohort B to validate the predictive signature obtained in cohort A, we found a 100% sensitivity but a low specificity. When using cohort A to validate the predictive signature of cohort B, we found an overall accuracy of around 70%.

We also carried out an analysis of the microarray data from cohorts A and B combined. For both cohorts combined, the number of gene probes used for prediction was reduced from an initial panel of twenty to five gene probes. The defined five gene signature panel included TNFRSF11B, STC1, PTGS2, IL-13R(alpha)2 and IL-11 and predicted the response to Infliximab therapy with 89% accuracy. All five proteins encoded by the five genes identified in this study are involved in signaling in the adaptive immune response, inflammation and TNF pathways(13-18). We confirmed the predictive value of each of the top five probes derived from the combined analysis by qPCR. Further study of the genes and pathways identified in the present studies should allow a better understanding of the molecular mechanisms of Infliximab action and mechanisms of resistance to anti-TNF therapy.

We propose to further prospectively validate this gene signature in patients to whom a first anti-TNF treatment is given.

In conclusion, we developed gene expression profiles in mucosal biopsies from two cohorts of Infliximab-naive patients prior to Infliximab therapy. Our studies demonstrate that a limited number of genes involved in the inflammatory cascade account for resistance of UC to respond to anti-TNFα therapy.

### Cloning, Expression and Purification of a TNFα-IL-13RA2 Diabody:

The invention provides a dual-specific ligand comprising a first immunoglobulin variable domain binding to TNFα and a second immunoglobulin variable domain binding to IL-13RA2.

Bispecific antibodies comprising complementary pairs of V_{H} and V_{L} regions are known in the art. These bispecific antibodies must comprise two pairs of V_{H} and V_{L}, each V_{H}/V_{L} pair binding to a single antigen or epitope.

The methods described involve hybrid hybridomas (Milstein & Cuello A C, Nature 305:537-40), minibodies (Hu et al. (1996), Cancer Res. 56:3055-3061), diabodies (Holliger et al. (1993), Proc. Natl. Acad. Sci. USA 90, 6444-6448; WO 94/13804), chelating recombinant antibodies (CRAbs) (Neri et al. (1995), J. Mol. Biol. 246, 367-373), biscFv (e.g., Atwell et al. (1996), Mol. Immunol. 33, 1301-1312), “knobs in holes” stabilized antibodies (Carter et al. (1997), Protein Sci. 6, 781-788).

Here, the bispecific diabody is produced by expressing two polypeptide chains with the structure V_{H}A-V_{L}B and V_{H}B-V_{L}A within the same cell in a diabody format. The small linker connecting the V_{H} and V_{L} domain to approximately five residues forces dimerization of the two polypeptide chains by crossover pairing of the V_{H} and V_{L} domains.

### Purification of RNA and Amplification of Variable Domains:

Total RNA from 10⁶ cells of the mouse hybridoma anti-TNFα (A) and IL-13RA2 (B) was extracted using the RNEASY® mini kit (Qiagen). mRNA was isolated from the total RNA preparation with the OLIGOTEX® mRNA kit (Qiagen GMBH Corporation, Germany). The complementary DNA (cDNA) was synthesized using an RT-PCR Kit (Roche Diagnostics) using oligo dT as primer. The polymerase chain reaction technique (PCR) for the specific amplification of the heavy and light chain variable domain genes was used. The employed synthetic primers were designed on the basis of the consensus sequences for mouse IgG and kappa chains, reported by E. Kabat et al. (U.S. Department of Health and Human Services, NIH, 1991).

For PCR, the following conditions are used: denaturizing to 94° C., one minute, annealing to 55° C., one minute, extension to 72° C., one minute, 25 cycles, with five additional minutes of extension to the temperature already described in the last cycle, everything in an Eppendorf Mastercycle machine. The final volumes of each reaction were 100 μL. All of the oligonucleotides were used to a final concentration of 1 μM.

The DNA amplified fragments were purified from agarose gels using the QIAQUICK® Gel Extraction Kit (Qiagen), and were cloned independently in the pPCR-script vector (Stratagene).

### Nucleotide Sequence of the Variable Domains:

The nucleotide sequence of the light and heavy chain variable domains cloned in the pPCR-script vector is determined by means of automated methods using commercially available kits.

pPCR-script derived plasmids, pV_{H}A, pV_{L}A and pV_{H}B, pV_{H}B, are selected as containing the correct sequence for the V_{H} and V_{L} of anti-TNF (A) and V_{H} and V_{L} of anti-IL-13RA2 (B), respectively.

### Assembly of the Diabody:

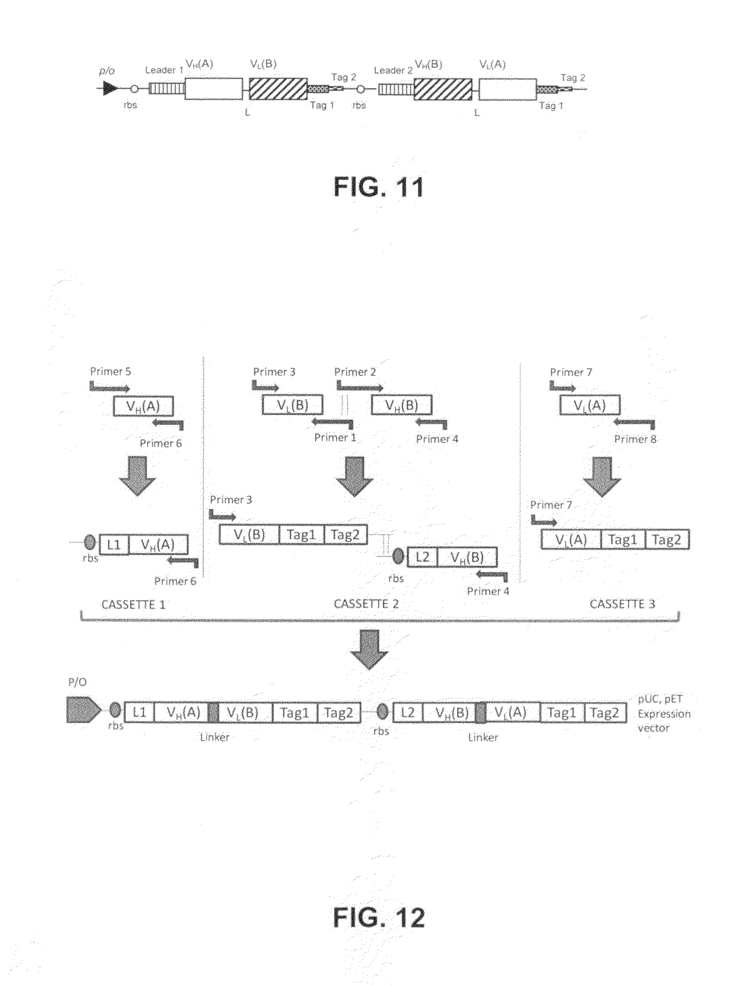

Different expression plasmids are available and derived from commercially available vectors such as pUC, pET28, pET20b, etc. Plasmids should only allow the construction of the following cassette (FIG. 11). To obtain this final cassette allowing expression of the diabody in a bacterial host cell, a multistep cloning procedure is necessary. Many different approaches are possible. An example is given below (FIG. 12).

Assembly of the V_{L}(B) and V_{H}(B) domains was performed by SOE-PCR (splicing by overlap extension) (McGuinness et al., 1996; Clackson et al., 1991). In short: Final V_{L}(B) and V_{H}(B) PCR products are assembled by nine PCR cycles without, followed by 25 cycles with pull-through primers 3 (containing linker (AKTTPKLGG (SEQ ID NO:)) encoding sequence) and 4 (containing linker encoding sequence) (FIG. 12). Primers 1 and 2 are partially complementary and comprise sequences encoding Tag 1, Tag 2 and an appropriate restriction site (primer 1) and sequence encoding an rbs, leader sequence and an appropriate restriction site (primer 2). Double digestion of the resulting assembled PCR product yields cassette 2.

V_{H}(A) and V_{L}(A) domains are PCR amplified from pHA and pVA, respectively, using primer 5 (containing rbs, leader sequence and appropriate restriction site) and primer 6 (containing linker encoding sequence and identical restriction site as in primer 3) and primer 7 (containing linker encoding sequence and identical restriction site as primer 4) and primer 8 (containing sequences encoding Tag 1 and Tag 2 and an appropriate restriction site), respectively. Double digestion of the obtained PCR products yields cassette 1 [V_{H}(A)] and cassette 3 [V_{L}(A)], respectively.

Cassettes 1, 2 and 3 are ligated together in the respective expression vector downstream of the promoter and operator site to driving secretion and subsequent assembly of the diabody in the periplasmic space.

### Diabody Expression and Purification:

For functional expression of the bispecific diabody in the bacterial periplasm, the obtained expression vector encoding the TNFα-IL-13RA2 diabody is transformed into E. coli K12 strain RV308 (ΔlacX74galISII::OP308strA). Transformed bacteria are grown overnight in shake flasks containing 2YT medium with 0.1 g/L ampicillin and 100 mM glucose (2YT_{GA}) at 26° C. Dilutions (1/50) of the overnight cultures in 2YT_{GA} are grown as flask cultures at 26° C. with shaking at 200 rpm. At OD₆₀₀=0.7, bacteria are harvested by centrifugation and resuspended in the same volume of YTBS medium (2YT containing 1 M sorbitol and 2.5 mM glycine betaine). Isopropyl β-D-thiogalactoside is added to a final concentration of 0.2 mM, and growth was continued at 23° C. for 13 hours. The bacterial cells are then harvested by centrifugation, and periplasmic extracts were isolated as previously described.

The periplasmic extract is passed through a filter of pore size 0.2 μm and dialyzed against 20 mmol/L Tris-HCl, 0.5 mol/L NaCl, pH 7.9 and purified as described before.

Due to the presence of a 6His Tag, purification is achieved by immobilized metal affinity chromatography (IMAC) on Ni²⁺-charged chelating agarose (GE Healthcare) following the manufacturer's recommendations.

The term “pharmaceutically acceptable” is used herein to mean that the modified noun is appropriate for use in a pharmaceutical product.

As used herein, the term “pharmaceutically acceptable carrier” also includes any and all solvents, dispersion media, coatings, antibacterial and antifungal agents, isotonic agents, absorption delaying agents, and the like. The use of such media and agents for pharmaceutically active substances is well known in the art. Except insofar as any conventional media or agent is incompatible with the compositions of this invention; its use in the therapeutic formulation is contemplated. Supplementary active ingredients can also be incorporated into the pharmaceutical formulations.

The term “treatment” refers to any process, action, application, therapy, or the like, wherein a mammal, including a human being, is subject to medical aid with the object of improving the mammal's condition, directly or indirectly. In the current invention, “treatment” also refers to prevention of an IBD, for instance, an UC or a CD.

Suppression means that IL-13R(alpha)2 activation, IL-13R(alpha)2 activity or IL-13R(alpha)2 expression occurs for at least 20%, 30%, 40%, 50%, 60%, 70%, 80%, 90% or even 100% less in the treated mammal compared with the mammal not treated with an inhibitor of IL-13R(alpha)2 of the invention.

The invention provides the use of a compound that inhibits the expression and/or activity of an IL-13R(alpha)2 for the manufacture of a medicament for increasing the efficiency an anti-TNFα treatment or IBD, in particular, UC or CD.

The term “a compound that inhibits the expression” refers here to gene expression and thus to the inhibition of gene transcription and/or translation of a gene transcript (mRNA), such as, for example, the IL-13R(alpha)2 or IL-13R(alpha)2 mRNA. Preferably, this inhibition is at least 20%, 30%, 40%, 50%, 60%, 70%, 80%, 90% or even higher. The term “a compound that inhibits the activity” refers here to the protein that is produced, such as the IL-13R(alpha)2 protein. This inhibition of activity leads to a diminished interaction of IL-13R(alpha)2 with its substrates, and diminished IL-13R(alpha)2 activity while under the catalyzed DNA degradation and an inhibition of the IL-13R(alpha)2 dependent inhibition of responding on an anti-TNFα IBD treatment. Preferably, the inhibition of IL-13R(alpha)2 is at least 20%, 30%, 40%, 50%, 60%, 70%, 80%, 90% or even higher.

The present disclosure shows that response to anti-TNFα in an IBD treatment significantly increased if IL-13R(alpha)2 is inhibited and that response to an anti-TNF treatment can be increased by the usage of inhibitors of IL-13R(alpha)2.

Thus, more specifically, the invention also relates to molecules that neutralize the activity of IL-13R(alpha)2 by interfering with its synthesis, translation, dimerization, or substrate-binding. By “molecules,” it is meant peptides, peptide aptamers, tetrameric peptides, proteins, organic molecules, soluble substrates of IL-13R(alpha)2 and any fragment or homologue thereof having the same neutralizing effect as stated above. Also, in the present invention, the molecules comprise antagonists of IL-13Ralpha2, such as anti- IL-13R(alpha)2 antibodies and functional fragments derived thereof, anti-sense RNA and DNA molecules and ribozymes that function to inhibit the translation of IL-13R(alpha)2, all capable of interfering and/or inhibiting the IL-13Ralpha2-dependent pathways.

By “synthesis” is meant transcription of IL-13R(alpha)2. Small molecules can bind on the promoter region of IL-13R(alpha)2 and inhibit binding of a transcription factor or the molecules can bind the transcription factor and inhibit binding to the IL-13R(alpha)2 promoter.

By “IL-13R(alpha)2” is also meant its isoforms, which occur as a result of alternative splicing, and allelic variants thereof.

Antagonists of IL-13R(alpha)2 can increase the anti-TNFα treatment response in IBD and, in particular, in UC or CD.

The term “antibody” or “antibodies” relates to an antibody characterized as being specifically directed against IL-13R(alpha)2 or any functional derivative thereof, with the antibodies being preferably monoclonal antibodies, or an antigen-binding fragment thereof, of the F(ab′)2, F(ab) or single chain Fv type, of the single domain antibody type or any type of recombinant antibody derived thereof. These antibodies of the invention, including specific polyclonal antisera prepared against of IL-13R(alpha)2, or any functional derivative thereof, have no cross-reactivity to others proteins. The monoclonal antibodies of the invention can, for instance, be produced by any hybridoma liable to be formed according to classical methods from splenic cells of an animal, particularly of a mouse or rat immunized against of IL-13R(alpha)2 or any functional derivative thereof, and of cells of a myeloma cell line, and to be selected by the ability of the hybridoma to produce the monoclonal antibodies recognizing of IL-13R(alpha)2 or any functional derivative thereof that have been initially used for the immunization of the animals. The monoclonal antibodies according to this embodiment of the invention may be humanized versions of the mouse monoclonal antibodies made by means of recombinant DNA technology, departing from the mouse and/or human genomic DNA sequences coding for H and L chains or from cDNA clones coding for H and L chains. Alternatively, the monoclonal antibodies according to this embodiment of the invention may be human monoclonal antibodies. Such human monoclonal antibodies are prepared, for instance, by means of human peripheral blood lymphocytes (PBL) repopulation of severe combined immune deficiency (SCID) mice as described in PCT/EP 99/03605 or by using transgenic non-human animals capable of producing human antibodies as described in U.S. Pat. No. 5,545,806. In addition, fragments derived from these monoclonal antibodies such as Fab, F(ab)′2 and ssFv (“single chain variable fragment”), providing they have retained the original binding properties, form part of the present invention. Such fragments are commonly generated by, for instance, enzymatic digestion of the antibodies with papain, pepsin, or other proteases. It is well known to the person skilled in the art that monoclonal antibodies, or fragments thereof, can be modified for various uses. The antibodies involved in the invention can be labeled by an appropriate label of the enzymatic, fluorescent, or radioactive type.

Small molecules, e.g., small organic molecules, and other drug candidates can be obtained, for example, from combinatorial and natural product libraries.

Random peptide libraries, such as the use of tetrameric peptide libraries such as described in WO0185796, consisting of all possible combinations of amino acids attached to a solid phase support, or such as a combinatorial library of peptide aptamers, which are proteins that contain a conformationally constrained peptide region of variable sequence displayed from a scaffold as described in Colas et al., Nature 380: 548-550, 1996, and Geyer et al., Proc. Natl. Acad. Sci. USA 96: 8567-8572, 1999, may be used in the present invention.

In addition, transdominant-negative mutant forms of IL-13R(alpha)2 ligands can be used to inhibit of IL-13Ralpha2-dependent pathways.

Also within the scope of the invention is the use of oligoribonucleotide sequences that include anti-sense RNA and DNA molecules and ribozymes that function to inhibit the translation of IL-13R(alpha)2 mRNA. Anti-sense RNA and DNA molecules act to directly block the translation of mRNA by binding to targeted mRNA and preventing protein translation. In regard to antisense DNA, oligodeoxyribonucleotides may be derived from the translation initiation site.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage. Within the scope of the invention are engineered hammerhead motif ribozyme molecules that specifically and efficiently catalyze endonucleolytic cleavage of IL-13R(alpha)2 sequences. Specific ribozyme cleavage sites within any potential RNA target are initially identified by scanning the target molecule for ribozyme cleavage sites that include the following sequences, GUA, GUU and GUC. Once identified, short RNA sequences of between 15 and 20 ribonucleotides corresponding to the region of the target gene containing the cleavage site may be evaluated for predicted structural features such as a secondary structure that may render the oligonucleotide sequence unsuitable.

Both anti-sense RNA and DNA molecules and ribozymes of the invention may be prepared by any method known in the art for the synthesis of RNA molecules. These include techniques for chemically synthesizing oligodeoxyribonucleotides well known in the art, such as, for example, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters, such as the T7 or SP6 polymerase promoters. Alternatively, antisense cDNA constructs that synthesize anti-sense RNA constitutively or inducibly, depending on the promoter used, can be introduced stably into cell lines.

The present invention provides an in vitro method of diagnosing for predicting if a subject suffering from an inflammatory condition of the large intestine and/or small intestine will respond to an anti-TNFα therapy, such method comprising: (a) analyzing the level of IL-13R(alpha)2 expression or activity of expression product in a biological sample isolated from the subject, and (b) comparing the level of expression or activity with the 13Ralpha2 expression or activity in a control sample; whereby a different level of IL-13R(alpha)2 expression or activity relative to a control sample is an indication of response to anti-TNFα therapy or a propensity thereto. A decreased level of IL-13R(alpha)2 is indicative of a positive response thereto. The in vitro method of diagnosing for predicting if a subject suffering from an inflammatory condition of the large intestine and/or small intestine will respond to an anti-TNFα therapy is particularly suitable for patients affected by IBD, such CD or UC.

In particular the present invention provides an in vitro method of diagnosing for predicting if a subject suffering from an inflammatory condition of the large intestine and/or small intestine will respond to an anti-TNFα antibody therapy, such as a therapy with an anti-TNFα therapeutic antibody, for instance, an antibody that blocks the action of TNFα by preventing it from binding to its receptor in the cell, such as Infliximab, Adalimumab or Etanercept, such method comprising: (a) analyzing the level of IL-13R(alpha)2 expression or activity of expression product in a biological sample isolated from the subject, and (b) comparing the level of expression or activity with the 13Ralpha2 expression or activity in a control sample; whereby a different level of IL-13R(alpha)2 expression or activity relative to a control sample is an indication of response to anti-TNFα therapy or a propensity thereto. A decreased level of IL-13R(alpha)2 is indicative of a positive response thereto and is predictive for the responders.

In yet a more specific embodiment of the present invention, an in vitro method of diagnosing for predicting if a subject suffering from an ulcerative colitis will respond to an anti-TNFα antibody therapy, such as a therapy with an anti-TNFα therapeutic antibody, for instance, an antibody that blocks the action of TNFα by preventing it from binding to its receptor in the cell, such as Infliximab, Adalimumab or Etanercept, such method comprising: (a) analyzing the level of IL-13R(alpha)2 expression or activity of expression product in a biological sample isolated from the subject, and (b) comparing the level of expression or activity with the 13Ralpha2 expression or activity in a control sample, whereby a different level of IL-13R(alpha)2 expression or activity relative to a control sample is an indication of response to anti-TNFα therapy or a propensity thereto. A decreased level of IL-13R(alpha)2 is indicative of a positive response thereto and is indicative for the responders.

The present invention furthermore concerns an in vitro method of diagnosis to predict the responding or non-responding of a subject on an anti-TNFα treatment of IBD, or a propensity thereto in a subject, the method comprising: (a) obtaining an expression profile in a biological sample isolated from the subject, wherein the expression profile consists of the analysis of the level of IL-13R(alpha)2 expression or activity of an IL-13R(alpha)2 expression product in combination with the gene expression level or activity of a gene product of at least one gene selected from the group consisting of TNFRSF11B, STC1, PTGS2 and IL-11; and (b) comparing the obtained expression profile to a reference expression profile to determine whether the sample is from the subject having an inflammatory bowel disease phenotype or a propensity thereto. In this in vitro method, the expression profile can consist of any one of the following combinations: IL-13R(alpha)2 and TNFRSF11B; IL-13R(alpha)2 and STC1; IL-13R(alpha)2 and PTGS2; IL-13R(alpha)2 and IL-11; IL-13R(alpha)2 and STC1 and PTGS2; IL-13R(alpha)2 and TNFRSF11B and PTGS2; IL-13R(alpha)2 and TNFRSF11B and STC1; IL-13R(alpha)2 and IL-11 and TNFRSF11B; IL-13R(alpha)2 and IL-11 and STC1; IL-13R(alpha)2 and IL-11 and PTGS2; IL-13R(alpha)2 and TNFRSF11B and PTGS2 and STC1; IL-13R(alpha)2 and IL-11 and PTGS2 and STC1; IL-13R(alpha)2 and TNFRSF11B and IL-11 and STC1; IL-13R(alpha)2 and TNFRSF11B and PTGS2 and IL-11.

The present invention furthermore concerns an in vitro method of diagnosis to predict the responding or non-responding of a subject on an anti-TNFα treatment of IBD, or a propensity thereto in a subject, the method comprising: (a) obtaining an expression profile in a biological sample isolated from the subject, wherein the expression profile consists of the analysis of the level of IL-13R(alpha)2 expression or activity of an IL-13R(alpha)2 expression product in combination with the gene expression level or activity of a gene product of at least two genes selected from the group consisting of TNFRSF11B, STC1, PTGS2 and IL-11; and (b) comparing the obtained expression profile to a reference expression profile to determine whether the sample is from a subject having an inflammatory bowel disease phenotype or a propensity thereto.

The present invention furthermore concerns an in vitro method of diagnosis to predict the responding or non-responding of a subject on an anti-TNFα treatment of IBD, or a propensity thereto in a subject, the method comprising: (a) obtaining an expression profile in a biological sample isolated from the subject, wherein the expression profile consists of the analysis of the level of IL-13R(alpha)2 expression or activity of an IL-13R(alpha)2 expression product in combination with the gene expression level or activity of a gene product of at least three genes selected from the group consisting of TNFRSF11B, STC1, PTGS2 and IL-11; and (b) comparing the obtained expression profile to a reference expression profile to determine whether the sample is from a subject having an inflammatory bowel disease phenotype or a propensity thereto.

The present invention furthermore concerns an in vitro method of diagnosis to predict the responding or non-responding of a subject on an anti-TNFα treatment of IBD, or a propensity thereto in a subject, the method comprising: (a) obtaining an expression profile in a biological sample isolated from the subject, wherein the expression profile consists of the analysis of the level of IL-13R(alpha)2 expression or activity of an expression product of the gene cluster of the genes IL-13R(alpha)2, TNFRSF11B, STC1, PTGS2 and IL-11; and (b) comparing the obtained expression profile to a reference expression profile to determine whether the sample is from a subject having an inflammatory bowel disease phenotype or a propensity thereto.

The expression product of any of the previously described in vitro methods can be a nucleic acid molecule selected from the group consisting of mRNA and cDNA mRNA or derived polypeptides. The sample in any of the previously described in vitro methods can be isolated from the subject and is selected from a group consisting of: (a) a liquid containing cells; (b) a tissue sample; (c) a cell sample; and (d) a cell biopsy, for instance, obtainable by a colonic mucosal biopsy.

The in vitro method according to any one of the previous methods hereinabove described can, in a particular embodiment, comprise the detection of the level of the nucleic acids or polypeptides carried out using at least one binding agent specifically binding to the nucleic acids or polypeptides to be detected. The binding agent can be detectably labeled. The label can be selected from the group consisting of a radioisotope, a bioluminescent compound, a chemiluminescent compound, a fluorescent compound, a metal chelate, biotin, digoxygenin and an enzyme. In a particular specific situation of this embodiment, at least one binding agent is an aptamer or an antibody selected from a group comprising: (a) a monoclonal antibody; (b) a polyclonal antibody; (c) a Fab-Fragment; (d) a single chain antibody; and (e) an antibody variable domain sequence; and the detection can furthermore comprise an immuno-cytochemical detection procedure.

In a particular specific situation of this embodiment, at least one binding agent, being a nucleic acid hybridizing to a nucleic acid, is used for the detection of the marker molecules, in particular, for the detection of IL-13R(alpha)2, TNFRSF11B, STC1, PTGS2 and IL-11 expression. Such method can further comprise the detection reaction comprising a nucleic acid amplification reaction. The method can be used for in situ detection.

The present invention provides an in vitro method of diagnosing for predicting if a subject suffering of an inflammatory condition of the large intestine and/or small intestine will respond to an anti-TNFα therapy, such method comprises: (a) analyzing the level of IL-13R(alpha)2 expression or activity of expression product in a biological sample isolated from the subject, and (b) comparing the level of expression or activity with the 13Ralpha2 expression or activity in a control sample; whereby a different level of IL-13R(alpha)2 expression or activity relative to a control sample is an indication of response to anti-TNFα therapy or a propensity thereto. A decreased level of IL-13R(alpha)2 is indicative of a positive response thereto. The in vitro method of diagnosing for predicting if a subject suffering from an inflammatory condition of the large intestine and/or small intestine will respond to an anti-TNFα therapy is particularly suitable for patients affected by inflammatory bowel disease (IBD) such as Crohn's disease (CD) or ulcerative colitis (UC).

In particular, the present invention provides an in vitro method of diagnosing for predicting if a subject suffering from inflammatory bowel disease (IBD), such as Crohn's disease (CD) or ulcerative colitis (UC), will respond to an anti-TNFα antibody therapy, such as a therapy with an anti-TNFα therapeutic antibody, for instance, an antibody that blocks the action of TNFα by preventing it from binding to its receptor in the cell, such as Infliximab, Adalimumab or Etanercept, such method comprising: (a) analyzing the level of IL-13R(alpha)2 expression or activity of expression product in a biological sample isolated from the subject, and (b) comparing the level of expression or activity with the 13Ralpha2 expression or activity in a control sample; whereby a different level of IL-13R(alpha)2 expression or activity relative to a control sample is an indication of response to anti-TNFα therapy or a propensity thereto. A decreased level of IL-13R(alpha)2 is indicative of a positive response thereto and is predictive for the responders.

In yet a more specific embodiment, the present invention provides an in vitro method of diagnosing for predicting if a subject suffering from inflammatory bowel disease (IBD), such as Crohn's disease (CD) or ulcerative colitis (UC), will respond to an anti-TNFα antibody therapy, such as a therapy with an anti-TNFα therapeutic antibody, for instance, an antibody that blocks the action of TNFα by preventing it from binding to its receptor in the cell, such as Infliximab, Adalimumab or Etanercept, such method comprising: (a) analyzing the level of IL-13R(alpha)2 expression or activity of expression product in a biological sample isolated from the subject, and (b) comparing the level of expression or activity with the 13Ralpha2 expression or activity in a control sample; whereby a different level of IL-13R(alpha)2 expression or activity relative to a control sample is an indication of response to anti-TNFα therapy or a propensity thereto. A decreased level of IL-13R(alpha)2 is indicative of a positive response thereto and is indicative for the responders.

The present invention furthermore concerns an in vitro method of diagnosis to predict the responding or non-responding of a subject on an anti-TNFα treatment of inflammatory bowel disease (IBD), such as Crohn's disease (CD) or ulcerative colitis (UC), or a propensity thereto in a subject, the method comprising: (a) obtaining an expression profile in a biological sample isolated from the subject, wherein the expression profile consists of the analysis of the level of IL-13R(alpha)2 expression or activity of an IL-13R(alpha)2 expression product in combination with the gene expression level or activity of a gene product of at least one gene selected from the group consisting of TNFRSF11B, STC1, PTGS2 and IL-11; and (b) comparing the obtained expression profile to a reference expression profile to determine whether the sample is from a subject having a inflammatory bowel disease (IBD), such as Crohn's disease (CD) or ulcerative colitis (UC) phenotype or a propensity thereto. In this in vitro method, the expression profile can consist of any one of the following combinations: IL-13R(alpha)2 and TNFRSF11B; IL-13R(alpha)2 and STC 1; IL-13R(alpha)2 and PTGS2; IL-13R(alpha)2 and IL-11; IL-13R(alpha)2 and STC1 and PTGS2; IL-13R(alpha)2 and TNFRSF11B and PTGS2; IL-13R(alpha)2 and TNFRSF11B and STC1; IL-13R(alpha)2 and IL-11 and TNFRSF11B; IL-13R(alpha)2 and IL-11 and STC1; IL-13R(alpha)2 and IL-11 and PTGS2; IL-13R(alpha)2 and TNFRSF11B and PTGS2 and STC1; IL-13R(alpha)2 and IL-11 and PTGS2 and STC1; IL-13R(alpha)2 and TNFRSF11B and IL-11 and STC1; IL-13R(alpha)2 and TNFRSF11B and PTGS2 and IL-11.

The present invention furthermore concerns an in vitro method of diagnosis to predict the responding or non-responding of a subject on an anti-TNFα treatment of inflammatory bowel disease (IBD), such as Crohn's disease (CD) or ulcerative colitis (UC), or a propensity thereto in a subject, the method comprising: (a) obtaining an expression profile in a biological sample isolated from the subject, wherein the expression profile consists of the analysis of the level of IL-13R(alpha)2 expression or activity of an IL-13R(alpha)2 expression product in combination with the gene expression level or activity of a gene product of at least two genes selected from the group consisting of TNFRSF11B, STC1, PTGS2 and IL-11; and (b) comparing the obtained expression profile to a reference expression profile to determine whether the sample is from a subject having an inflammatory bowel disease phenotype or a propensity thereto.

The present invention furthermore concerns an in vitro method of diagnosis to predict the responding or non-responding of a subject on an anti-TNFα treatment of inflammatory bowel disease (IBD), such as Crohn's disease (CD) or ulcerative colitis (UC), or a propensity thereto in a subject, the method comprising: (a) obtaining an expression profile in a biological sample isolated from the subject, wherein the expression profile consists of the analysis of the level of IL-13R(alpha)2 expression or activity of an IL-13R(alpha)2 expression product in combination with the gene expression level or activity of a gene product of at least three genes selected from the group consisting of TNFRSF11B, STC1, PTGS2 and IL-11; and (b) comparing the obtained expression profile to a reference expression profile to determine whether the sample is from a subject having an inflammatory bowel disease (IBD), such as Crohn's disease (CD) or ulcerative colitis (UC) phenotype or a propensity thereto.

The present invention furthermore concerns an in vitro method of diagnosis to predict the responding or non-responding of a subject on an anti-TNFα treatment of inflammatory bowel disease (IBD), such as Crohn's disease (CD) or ulcerative colitis (UC), or a propensity thereto in a subject, the method comprising: (a) obtaining an expression profile in a biological sample isolated from the subject, wherein the expression profile consists of the analysis of the level of IL-13R(alpha)2 expression or activity of an expression product of the gene cluster of the genes IL-13R(alpha)2, TNFRSF11B, STC1, PTGS2 and IL-11; and (b) comparing the obtained expression profile to a reference expression profile to determine whether the sample is from a subject having an inflammatory bowel disease (IBD), such as Crohn's disease (CD) or ulcerative colitis (UC) phenotype or a propensity thereto.

The expression product of any of the previously described in vitro methods can be a nucleic acid molecule selected from the group consisting of mRNA and cDNA mRNA or derived polypeptides. The sample in any of the previously described in vitro methods can be isolated from the subject and is selected from a group consisting of: (a) a liquid containing cells; (b) a tissue sample; (c) a cell sample; and (d) a cell biopsy, for instance, obtainable by a colonic mucosal biopsy.

The in vitro method according to any one of the previous methods hereinabove described can, in a particular embodiment, comprise the detection of the level of the nucleic acids or polypeptides carried out using at least one binding agent specifically binding to the nucleic acids or polypeptides to be detected. The binding agent can be detectably labeled. The label can be selected from the group consisting of a radioisotope, a bioluminescent compound, a chemiluminescent compound, a fluorescent compound, a metal chelate, biotin, digoxygenin and an enzyme. In a particular specific situation of this embodiment, at least one binding agent is an aptamer or an antibody selected from a group comprising: (a) a monoclonal antibody; (b) a polyclonal antibody; (c) a Fab-Fragment; (d) a single chain antibody; and (e) an antibody variable domain sequence; and the detection can furthermore comprise an immuno-cytochemical detection procedure.

In a particular specific situation of this embodiment, at least one binding agent being a nucleic acid hybridizing to a nucleic acid is used for the detection of the marker molecules, in particular, for the detection of IL-13R(alpha)2, TNFRSF11B, STC1, PTGS2 and IL-11 expression. Such method can further comprise the detection reaction comprising a nucleic acid amplification reaction. The method can be used for in situ detection.

Another embodiment of the present invention is a diagnostic test kit for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of ulcerative colitis in patients receiving an anti-TNFα therapy comprising: (a) a predetermined amount of an antibody specific for IL-13R(alpha)2; (b) a predetermined amount of a specific binding partner to the antibody; (c) buffers and other reagents necessary for monitoring detection of antibody bound to IL-13R(alpha)2; and wherein, either the antibody or the specific binding partner are detectably labeled. This diagnostic kit can furthermore comprise directions for use of the kit.

Another embodiment of the present invention is a diagnostic test kit for use in diagnosing a subject for responsiveness on an anti-TNFα treatment of ulcerative colitis, or for use in monitoring the effectiveness of therapy of inflammatory bowel disease (IBD), such as Crohn's disease (CD) or ulcerative colitis (UC), in patients receiving an anti-TNFα therapy comprising: (a) a predetermined amount of an antibody specific for TNFRSF11B; (b) a predetermined amount of a specific binding partner to the antibody; (c) buffers and other reagents necessary for monitoring detection of antibody bound to TNFRSF11B; and wherein, either the antibody or the specific binding partner are detectably labeled. This diagnostic kit can furthermore comprise directions for use of the kit.

Another embodiment of the present invention is a diagnostic test kit for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) a predetermined amount of an antibody specific for STC 1; (b) a predetermined amount of a specific binding partner to the antibody; (c) buffers and other reagents necessary for monitoring detection of antibody bound to STC1; and wherein, either the antibody or the specific binding partner are detectably labeled. This diagnostic kit can furthermore comprise directions for use of the kit.

Another embodiment of the present invention is a diagnostic test kit for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) a predetermined amount of an antibody specific for PTGS2; (b) a predetermined amount of a specific binding partner to the antibody; (c) buffers and other reagents necessary for monitoring detection of an antibody bound to PTGS2; and wherein, either the antibody or the specific binding partner are detectably labeled. This diagnostic kit can furthermore comprise directions for use of the kit.

Another embodiment of the present invention is a diagnostic test kit for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) a predetermined amount of an antibody specific for IL-11; (b) a predetermined amount of a specific binding partner to the antibody; (c) buffers and other reagents necessary for monitoring detection of antibody bound to IL-11; and wherein, either the antibody or the specific binding partner are detectably labeled. This diagnostic kit can furthermore comprise directions for use of the kit.

Another embodiment of the present invention is a diagnostic test kit for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) a predetermined amount of a predetermined amount of two different antibodies, each specific for two different proteins of the group IL-13R(alpha)2, TNFRSF11B, STC1, PTGS2 and IL-11; (b) a predetermined amount of a specific binding partner to the antibody; (c) buffers and other reagents necessary for monitoring detection of antibody bound to the selected proteins of the IL-13R(alpha)2, TNFRSF11B, STC1, PTGS2 and IL-11; and wherein, either the antibody or the specific binding partner are detectably labeled. This diagnostic kit can furthermore comprise directions for use of the kit.

Another embodiment of the present invention is a diagnostic test kit for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) a predetermined amount of a predetermined amount of three different antibodies, each specific for three different proteins of the group IL-13R(alpha)2, TNFRSF11B, STC1, PTGS2 and IL-11; (b) a predetermined amount of a specific binding partner to the antibody; (c) buffers and other reagents necessary for monitoring detection of an antibody bound to the selected proteins of the IL-13R(alpha)2, TNFRSF11B, STC1, PTGS2 and IL-11; and wherein, either the antibody or the specific binding partner are detectably labeled. This diagnostic kit can furthermore comprise directions for use of the kit.

Another embodiment of the present invention is a diagnostic test kit for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) a predetermined amount of four different antibodies, each specific for four different proteins of the group IL-13R(alpha)2, TNFRSF11B, STC1, PTGS2 and IL-11; (b) a predetermined amount of a specific binding partner to the antibody; (c) buffers and other reagents necessary for monitoring detection of antibody bound to the selected proteins of the IL-13R(alpha)2, TNFRSF11B, STC1, PTGS2 and IL-11; and wherein, either the antibody or the specific binding partner are detectably labeled. This diagnostic kit can furthermore comprise directions for use of the kit.

Another embodiment of the present invention is a diagnostic test kit for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) a predetermined amount of an antibody specific for each of the proteins of the group consisting of IL-13R(alpha)2, TNFRSF11B, STC1, PTGS2 and IL-11; (b) a predetermined amount of a specific binding partner to the antibody; (c) buffers and other reagents necessary for monitoring detection of antibody bound to the selected proteins of the IL-13R(alpha)2, TNFRSF11B, STC1, PTGS2 and IL-11; and wherein, either the antibody or the specific binding partner are detectably labeled. This diagnostic kit can furthermore comprise directions for use of the kit.

Another embodiment of the present invention is for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) a nucleic acid encoding the 13Ralpha2 protein ; (b) reagents useful for monitoring the expression level of the one or more nucleic acids or proteins encoded by the nucleic acids of step (a); and (c) instructions for use of the kit.

Another embodiment of the present invention is for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) a nucleic acid encoding the TNFRSF11B protein ; (b) reagents useful for monitoring the expression level of the one or more nucleic acids or proteins encoded by the nucleic acids of step (a); and (c) instructions for use of the kit.

Another embodiment of present invention is for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) a nucleic acid encoding the STC1 protein; (b) reagents useful for monitoring the expression level of the one or more nucleic acids or proteins encoded by the nucleic acids of step (a); and (c) instructions for use of the kit.

Another embodiment of the present invention is for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC) in patients receiving an anti-TNFα therapy comprising: (a) a nucleic acid encoding the PTGS2 protein ; (b) reagents useful for monitoring the expression level of the one or more nucleic acids or proteins encoded by the nucleic acids of step (a); and (c) instructions for use of the kit.

Another embodiment of the present invention is for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) a nucleic acid encoding the IL-11 protein ; (b) reagents useful for monitoring the expression level of the one or more nucleic acids or proteins encoded by the nucleic acids of step (a); and (c) instructions for use of the kit.

Another embodiment of the present invention is for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of ulcerative colitis in patients receiving an anti-TNFα therapy comprising: (a) nucleic acids encoding the 13Ralpha2, TNFRSF11B, STC1, PTGS2 and IL-11 protein; (b) reagents useful for monitoring the expression level of the one or more nucleic acids or proteins encoded by the nucleic acids of step (a); and (c) instructions for use of the kit.

Another embodiment of the present invention is for use in diagnosing a subject for responsiveness on an anti-TNFα treatment of ulcerative colitis, or for use in monitoring the effectiveness of therapy of ulcerative colitis in patients receiving an anti-TNFα therapy comprising: (a) one or more nucleic acids encoding one or more of the proteins selected from the group consisting of 13Ralpha2, TNFRSF11B, STC1, PTGS2 and IL-11; (b) reagents useful for monitoring the expression level of the one or more nucleic acids or proteins encoded by the nucleic acids of step (a); and (c) instructions for use of the kit.

Another embodiment of the present invention is for use in diagnosing a subject for responsiveness on an anti-TNFα treatment of ulcerative colitis, or for use in monitoring the effectiveness of therapy of ulcerative colitis in patients receiving an anti-TNFα therapy comprising: (a) one or more nucleic acids encoding two or more of the proteins selected from the group consisting of 13Ralpha2, TNFRSF11B, STC1, PTGS2 and IL-11; (b) reagents useful for monitoring the expression level of the one or more nucleic acids or proteins encoded by the nucleic acids of step (a); and (c) instructions for use of the kit.

Another embodiment of the present invention is for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) one or more nucleic acids encoding three or more of the proteins selected from the group consisting of 13Ralpha2, TNFRSF11B, STC1, PTGS2 and IL-11; (b) reagents useful for monitoring the expression level of the one or more nucleic acids or proteins encoded by the nucleic acids of step (a); and (c) instructions for use of the kit.

Another embodiment of the present invention is for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) one or more nucleic acids encoding four or more of the proteins selected from the group consisting of 13Ralpha2, TNFRSF11B, STC1, PTGS2 and IL-11; (b) reagents useful for monitoring the expression level of the one or more nucleic acids or proteins encoded by the nucleic acids of step (a); and (c) instructions for use of the kit.

Another embodiment of the present invention is for use in diagnosing a subject for responsiveness on an anti-TNFα treatment of inflammatory bowel disease (IBD), such as Crohn's disease (CD) or ulcerative colitis (UC), or for use in monitoring the effectiveness of therapy of inflammatory bowel disease (IBD), such as Crohn's disease (CD) or ulcerative colitis (UC), in patients receiving an anti-TNFα therapy comprising: (a) nucleic acids encoding the proteins selected from the group consisting of 13Ralpha2, TNFRSF11B, STC1, PTGS2 and IL-11; (b) reagents useful for monitoring the expression level of the one or more nucleic acids or proteins encoded by the nucleic acids of step (a); and (c) instructions for use of the kit.

The present invention also comprises the following embodiments as expressed hereunder:

A pharmaceutical composition or pharmaceutical pack comprising an effective amount of an isolated compound that inhibits or that blocks, inhibits or suppresses the action expression and/or activity of IL-13R(alpha)2 and a compound that blocks, inhibits or suppresses the action of TNFα, such as Infliximab, Adalimumab or Etanercept, for use in a treatment to cure or to prevent inflammatory bowel disease.

This pharmaceutical composition, wherein the compound targeting IL-13R(alpha)2 is selected from the list consisting of a nucleotide, an antibody, a ribozyme, a tetrameric peptide, a peptide aptamer and a mutant IL-13R(alpha)2 protein.

Such a pharmaceutical composition wherein the nucleotide is an antisense DNA or RNA, siRNA, miRNA or an RNA or DNA aptamer.

Such a pharmaceutical composition wherein the antibody targeting IL-13R(alpha)2 is a monoclonal antibody or an antibody fragment specifically directed to IL-13R(alpha)2 or an antigen-binding fragment thereof.

Such a pharmaceutical composition wherein the antibody or antibody fragment is humanized.

Such a pharmaceutical composition wherein the anti-TNFα compound and the anti-IL-13R(alpha)2 compound are formulated separately and in individual dosage amounts.

Such a pharmaceutical composition wherein the anti-TNFα and the anti-IL-13R(alpha)2 is a diabody.

Such a pharmaceutical composition wherein the inflammatory bowel disease is a Crohn's disease.

9. The use of a compound having an inhibitory action on the IL-13R(alpha)2-activated pathway or that inhibits the expression and/or activity of IL-13R(alpha)2 in the manufacture of a medicament for the treatment of inflammatory bowel disease.

Such a use of claim 9, wherein the compound is selected from the list consisting of a nucleotide, an antibody, a ribozyme, a tetrameric peptide, a peptide aptamer, and a mutant IL-13R(alpha)2 protein.

Such a use wherein the nucleotide is an antisense DNA or RNA, siRNA, miRNA or an RNA or DNA aptamer.

Such a use wherein the compound is conjugated with a protein transduction domain.

Such a use wherein the medicament is for the treatment of an inflammatory bowel disease of the group consisting of Crohn's disease, ulcerative colitis, Collagenous colitis, Lymphocytic colitis, Ischaemic colitis, Diversion colitis, Behcet's syndrome, Infective colitis and Indeterminate colitis.

In another aspect, the invention relates to a pharmaceutical composition comprising the antibody, preferentially a human antibody, against 13Ralpha2 and/or the antibody, preferentially a human antibody, against TNFα of the invention as defined in any of the claims or embodiments herein and a pharmaceutically acceptable carrier. In another aspect, the pharmaceutical composition is in a form suitable for injection or infusion. In another aspect, the pharmaceutical composition is a liposome formulation. Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions. Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation.

It is especially advantageous to formulate parenteral compositions in dosage unit form for ease of administration and uniformity of dosage. “Dosage unit form” as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

### REFERENCES TO THIS APPLICATION

(1) Targan S R, Hanauer S B, Van Deventer S J et al. A short-term study of chimeric monoclonal antibody cA2 to tumor necrosis factor alpha for Crohn's disease. Crohn's Disease cA2 Study Group. N. Engl. J. Med. 1997; 337(15):1029-1035.
(2) Rutgeerts P, Feagan B G, Lichtenstein G R et al. Comparison of scheduled and episodic treatment strategies of infliximab in Crohn's disease. Gastroenterology 2004; 126(2):402-413.
(3) Present D H, Rutgeerts P, Targan S et al. Infliximab for the treatment of fistulas in patients with Crohn's disease. N. Engl. J. Med. 1999; 340(18):1398-1405.
(4) Sands B E, Anderson F H, Bernstein C N et al. Infliximab maintenance therapy for fistulizing Crohn's disease. N. Engl. J. Med. 2004; 350(9):876-885.
(5) Rutgeerts P, Sandborn W J, Feagan B G et al. Infliximab for induction and maintenance therapy for ulcerative colitis. N. Engl. J. Med. 2005; 353(23):2462-2476.
(6) Schena M, Shalon D, Davis R W, Brown P O. Quantitative monitoring of gene expression patterns with a complementary DNA microarray. Science 1995; 270(5235):467-470.
(7) Bullinger L, Dohner K, Bair E et al. Use of gene-expression profiling to identify prognostic subclasses in adult acute myeloid leukemia. N. Engl. J. Med. 2004; 350(16):1605-1616.
(8) Heller R A, Schena M, Chai A et al. Discovery and analysis of inflammatory disease-related genes using cDNA microarrays. Proc. Natl. Acad. Sci. U.S.A. 1997; 94(6):2150-2155.
(9) D'Haens G R, Geboes K, Peeters M, Baert F, Penninckx F, Rutgeerts P. Early lesions of recurrent Crohn's disease caused by infusion of intestinal contents in excluded ileum. Gastroenterology 1998; 114(2):262-267.
(10) Geboes K, Riddell R, Ost A, Jensfelt B, Persson T, Lofberg R. A reproducible grading scale for histological assessment of inflammation in ulcerative colitis. Gut 2000; 47(3):404-409.
(11) Gentleman R C, Carey V J, Bates D M et al. Bioconductor: open software development for computational biology and bioinformatics. Genome Biol. 2004; 5(10):R80.
(12) Irizarry R A, Hobbs B, Collin F et al. Exploration, normalization, and summaries of high density oligonucleotide array probe level data. Biostatistics 2003; 4(2):249-264.
(13) Smyth G K. Linear models and empirical Bayes methods for assessing differential expression in microarray experiments. Stat. Appl. Genet. Mol. Biol. 2004; 3: Article 3.
(14) Tibshirani R, Hastie T, Narasimhan B, Chu G. Diagnosis of multiple cancer types by shrunken centroids of gene expression. Proc. Natl. Acad. Sci. U.S.A. 2002; 99(10):6567-6572.
(15) Benjamini Y, Hochberg Y. Controlling the false discovery rate: a practical and powerful approach to multiple testing. Journal of the Royal Statistical Society B 1995; 85:289-300.
(16) Dennis G, Jr., Sherman B T, Hosack D A et al. DAVID: Database for Annotation,

Visualization, and Integrated Discovery. Genome Biol. 2003; 4(5):3.
(17) Pfaffl M W. A new mathematical model for relative quantification in real-time RT-PCR. Nucleic Acids Res. 2001; 29(9):e45.
(18) Kioi M, Kawakami M, Shimamura T, Husain S R, Puri R K. Interleukin-13 receptor alpha2 chain: a potential biomarker and molecular target for ovarian cancer therapy. Cancer 2006; 107(6):1407-1418.
(19) Fichtner-Feigl S, Strober W, Kawakami K, Puri R K, Kitani A. IL-13 signaling through the IL-13alpha2 receptor is involved in induction of TGF-beta1 production and fibrosis. Nat. Med. 2006; 12(1):99-106.
(20) Fichtner-Feigl S, Fuss I J, Young C A et al. Induction of IL-13 triggers TGF-beta1 -dependent tissue fibrosis in chronic 2,4,6-trinitrobenzene sulfonic acid colitis. J. Immunol. 2007; 178(9):5859-5870.

TABLE 1
Baseline Characteristics of the patients

| UC patients (n = 24) | | | CD patients (n = 19) | | |
|---|---|---|---|---|---|
| Male/Female (%) | 14/10 | (58.3/41.7) | Male/Female (%) | 11/8 | (57.9/42.1) |
| Median (IQR) age at first infusion (years) | 41.4 | (32.3-50.9) | Median (IQR) age at first infusion (years) | 31.8 | (23.7-47.5) |
| Median (IQR) weight at first infusion (kg) | 72.5 | (67-80.3) | Median (IQR) weight at first infusion (kg) | 68 | (60.5-77.5) |
| Median (IQR) duration of disease | 7.3 | (2.7-17.1) | Median (IQR) duration of disease prior | 6.4 | (3.1-20.9) |
| prior to first IFX (years) | | | to first IFX (years) | | |
| Extent of disease | | | Extent of disease | | |
| Left-sided colitis (%) | 7 | (29.2) | Ileocolon (%) | 5 | (26.3) |
| Pancolitis (%) | 17 | (70.8) | Colon (%) | 14 | (73.7) |
| Median (IQR) C-reactive protein | 4 | (1.8-19.1) | Median (IQR) C-reactive protein at | 10.2 | (4.3-35) |
| at first IFX (mg/dL) | | | first IFX (mg/dL) | | |
| Concomitant medication at first IFX (%) | | | Concomitant medication at first IFX (%) | | |
| 5-Aminosalicylates | 18 | (75) | 5-Aminosalicylates | 8 | (42.1) |
| Corticosteroids | 7 | (29.2) | Corticosteroids | 4 | (21.1) |
| Azathioprine/6-Mercaptopurine | 15 | (62.5) | Azathioprine/6-Mercaptopurine | 14 | (73.7) |
| Methotrexate | 0 | (0) | Methotrexate | 0 | (0) |
| Corticosteroids + Immunosuppressants | 3 | (12.5) | Corticosteroids + Immunosuppressants | 2 | (10.5) |
| Active smoking at first IFX (%) | 2 | (8.3) | Active smoking at first IFX (%) | 6 | (31.6) |

SUPPLEMENTARY TABLE 2
GO analyses (EASE score <0.01 combined with counts (DE probe
sets involved in the biological process) >10) of the DΣ
probe sets and the downregulated DE probe sets between R and NR
from the LIMMA analyses in CD and IBD. The biological processes
are ranked based on the increasing order of the EASE score.

| | | |
|---|---|---|
| Biological Process | Count | EASE score |

GO analysis of DE probe sets between R and NR from LIMMA analysis in CD

| | | |
|---|---|---|
| immune response | 118 | 7.04E−39 |
| defense response | 120 | 4.42E−36 |
| response to biotic stimulus | 122 | 1.22E−35 |
| response to pest, pathogen or parasite | 80 | 6.12E−35 |
| response to wounding | 67 | 9.73E−34 |
| response to other organism | 80 | 4.84E−33 |
| response to external stimulus | 74 | 1.73E−32 |
| organismal physiological process | 161 | 9.29E−30 |
| inflammatory response | 45 | 9.18E−27 |
| response to stress | 97 | 7.14E−26 |
| response to stimulus | 157 | 1.21E−23 |
| cell adhesion | 62 | 4.58E−17 |
| cell communication | 165 | 2.87E−15 |
| humoral immune response | 29 | 4.86E−15 |
| signal transduction | 155 | 6.88E−15 |
| development | 104 | 1.59E−13 |
| organ development | 49 | 1.76E−13 |
| humoral defense mechanism (sensu Vertebrata) | 23 | 6.38E−13 |
| taxis | 23 | 2.14E−12 |
| chemotaxis | 23 | 2.14E−12 |
| cell proliferation | 47 | 3.96E−12 |
| locomotory behavior | 23 | 4.82E−12 |
| antimicrobial humoral response (sensu Vertebrata) | 18 | 1.12E−10 |
| behavior | 26 | 1.19E−10 |
| morphogenesis | 47 | 1.37E−10 |
| antimicrobial humoral response | 18 | 2.35E−10 |
| regulation of cell proliferation | 31 | 6.26E−10 |
| immune cell activation | 19 | 7.82E−10 |
| cell activation | 19 | 9.09E−10 |
| locomotion | 27 | 1.33E−09 |
| localization of cell | 27 | 1.33E−09 |
| cell motility | 27 | 1.33E−09 |
| angiogenesis | 15 | 2.99E−09 |
| blood vessel morphogenesis | 15 | 5.37E−09 |
| blood vessel development | 15 | 5.37E−09 |
| vasculature development | 15 | 5.37E−09 |
| organ morphogenesis | 24 | 2.18E−08 |
| positive regulation of cell proliferation | 19 | 2.45E−08 |
| cell differentiation | 38 | 3.75E−08 |
| negative regulation of biological process | 49 | 4.20E−08 |
| response to chemical stimulus | 32 | 8.47E−08 |
| negative regulation of cellular process | 46 | 8.78E−08 |
| response to abiotic stimulus | 33 | 6.29E−07 |
| phosphate transport | 15 | 7.41E−07 |
| cellular defense response | 15 | 7.41E−07 |
| lymphocyte activation | 14 | 1.07E−06 |
| positive regulation of biological process | 40 | 1.17E−06 |
| regulation of body fluids | 15 | 1.18E−06 |
| hemostasis | 14 | 1.96E−06 |
| inorganic anion transport | 18 | 2.58E−06 |
| cellular morphogenesis | 23 | 5.61E−06 |
| blood coagulation | 13 | 5.81E−06 |
| calcium ion homeostasis | 12 | 5.82E−06 |
| coagulation | 13 | 7.29E−06 |
| death | 36 | 8.82E−06 |
| positive regulation of physiological process | 31 | 1.27E−05 |
| metal ion homeostasis | 14 | 1.57E−05 |
| cell surface receptor linked signal transduction | 73 | 1.60E−05 |
| wound healing | 13 | 1.70E−05 |
| cell death | 35 | 1.89E−05 |
| apoptosis | 34 | 2.14E−05 |
| programmed cell death | 34 | 2.30E−05 |
| homeostasis | 18 | 2.61E−05 |
| cation homeostasis | 14 | 2.91E−05 |
| anion transport | 18 | 2.98E−05 |
| di-, tri-valent inorganic cation homeostasis | 13 | 3.66E−05 |
| positive regulation of cellular physiological process | 29 | 4.15E−05 |
| cell ion homeostasis | 14 | 4.78E−05 |
| negative regulation of physiological process | 37 | 5.77E−05 |
| positive regulation of cellular process | 32 | 6.04E−05 |
| regulation of immune response | 11 | 6.28E−05 |
| regulation of development | 11 | 6.28E−05 |
| skeletal development | 14 | 7.62E−05 |
| negative regulation of cellular physiological process | 35 | 1.41E−04 |
| cell-cell signaling | 31 | 1.42E−04 |
| intracellular signaling cascade | 52 | 1.54E−04 |
| ion homeostasis | 14 | 1.56E−04 |
| regulation of organismal physiological process | 14 | 1.67E−04 |
| hemopoietic or lymphoid organ development | 11 | 1.67E−04 |
| negative regulation of cell proliferation | 14 | 2.64E−04 |
| cell homeostasis | 14 | 3.19E−04 |
| cell migration | 11 | 4.56E−04 |
| regulation of apoptosis | 21 | 0.001521459 |
| regulation of programmed cell death | 21 | 0.001622414 |
| protein kinase cascade | 18 | 0.001703466 |
| tissue development | 13 | 0.005207525 |
| negative regulation of apoptosis | 11 | 0.005257505 |
| negative regulation of programmed cell death | 11 | 0.005516967 |
| second-messenger-mediated signaling | 13 | 0.006120657 |
| cell growth | 11 | 0.009884587 |
| regulation of cell size | 11 | 0.009884587 |

GO analysis of DE probe sets between R and NR from LIMMA analysis in IBD

| | | |
|---|---|---|
| immune response | 114 | 5.87E−38 |
| defense response | 117 | 5.19E−36 |
| response to biotic stimulus | 119 | 1.26E−35 |
| response to pest, pathogen or parasite | 78 | 1.28E−34 |
| response to other organism | 79 | 1.35E−33 |
| response to wounding | 65 | 4.75E−33 |
| organismal physiological process | 160 | 8.83E−32 |
| response to external stimulus | 71 | 3.34E−31 |
| inflammatory response | 44 | 1.61E−26 |
| response to stress | 95 | 3.97E−26 |
| response to stimulus | 153 | 5.67E−24 |
| humoral immune response | 32 | 1.99E−18 |
| taxis | 26 | 1.07E−15 |
| chemotaxis | 26 | 1.07E−15 |
| locomotory behavior | 26 | 2.83E−15 |
| cell communication | 158 | 1.15E−14 |
| humoral defense mechanism (sensu Vertebrata) | 24 | 2.77E−14 |
| signal traasduction | 144 | 1.04E−12 |
| cell adhesion | 52 | 5.13E−12 |
| behavior | 27 | 7.77E−12 |
| cell proliferation | 43 | 1.60E−10 |
| organ development | 42 | 3.94E−10 |
| antimicrobial humoral response (sensu Vertebrata) | 17 | 5.42E−10 |
| development | 91 | 1.00E−09 |
| antimicrobial humoral response | 17 | 1.09E−09 |
| response to chemical stimulus | 34 | 2.39E−09 |
| negative regulation of biological process | 49 | 1.05E−08 |
| immune cell activation | 17 | 2.14E−08 |
| cell activation | 17 | 2.43E−08 |
| negative regulation of cellular process | 45 | 6.66E−08 |
| phosphate transport | 16 | 6.69E−08 |
| response to abiotic stimulus | 34 | 7.40E−08 |
| cell-cell signaling | 37 | 1.74E−07 |
| cellular defense response | 15 | 4.40E−07 |
| morphogenesis | 38 | 8.12E−07 |
| inorganic anion transport | 18 | 1.43E−06 |
| regulation of cell proliferation | 24 | 3.37E−06 |
| homeostasis | 19 | 3.82E−06 |
| calcium ion homeostasis | 12 | 3.84E−06 |
| regulation of body fluids | 14 | 4.08E−06 |
| lymphocyte activation | 13 | 4.18E−06 |
| hemostasis | 13 | 7.26E−06 |
| innate immune response | 11 | 9.30E−06 |
| metal ion homeostasis | 14 | 9.86E−06 |
| positive regulation of cell proliferation | 15 | 1.08E−05 |
| wound healing | 13 | 1.10E−05 |
| positive regulation of biological process | 36 | 1.71E−05 |
| anion transport | 18 | 1.72E−05 |
| cation homeostasis | 14 | 1.85E−05 |
| blood coagulation | 12 | 2.19E−05 |
| di-, tri-valent inorganic cation homeostasis | 13 | 2.39E−05 |
| coagulation | 12 | 2.69E−05 |
| cell ion homeostasis | 14 | 3.05E−05 |
| regulation of immune response | 11 | 4.36E−05 |
| regulation of development | 11 | 4.36E−05 |
| locomotion | 19 | 4.61E−05 |
| localization of cell | 19 | 4.61E−05 |
| cell motility | 19 | 4.61E−05 |
| skeletal development | 14 | 4.90E−05 |
| cell differentiation | 30 | 5.34E−05 |
| negative regulation of physiological process | 36 | 5.41E−05 |
| cell surface receptor linked signal transduction | 68 | 7.85E−05 |
| intracellular signaling cascade | 51 | 9.96E−05 |
| ion homeostasis | 14 | 1.01E−04 |
| hemopoietic or lymphoid organ development | 11 | 1.17E−04 |
| tissue development | 16 | 1.17E−04 |
| death | 32 | 1.25E−04 |
| protein kinase cascade | 20 | 1.38E−04 |
| negative regulation of cellular physiological process | 34 | 1.38E−04 |
| cell homeostasis | 14 | 2.09E−04 |
| cell death | 31 | 2.48E−04 |
| apoptosis | 30 | 2.93E−04 |
| programmed cell death | 30 | 3.11E−04 |
| anti-apoptosis | 12 | 3.58E−04 |
| regulation of organismal physiological process | 13 | 4.16E−04 |
| organ morphogenesis | 16 | 5.12E−04 |
| positive regulation of physiological process | 26 | 5.66E−04 |
| regulation of kinase activity | 12 | 7.32E−04 |
| regulation of protein kinase activity | 12 | 7.32E−04 |
| positive regulation of cellular physiological process | 25 | 7.48E−04 |
| regulation of transferase activity | 12 | 7.79E−04 |
| positive regulation of cellular process | 28 | 7.86E−04 |
| cellular morphogenesis | 18 | 8.23E−04 |
| negative regulation of apoptosis | 12 | 0.001173889 |
| negative regulation of programmed cell death | 12 | 0.00124191 |
| regulation of enzyme activity | 16 | 0.002122662 |
| sensory perception | 32 | 0.003376446 |
| second-messenger-mediated signaling | 13 | 0.004398719 |
| regulation of apoptosis | 19 | 0.005040833 |
| regulation of programmed cell death | 19 | 0.005280844 |
| negative regulation of cell proliferation | 11 | 0.006494387 |

GO analysis of downregulated DE probe sets
between R and NR from LIMMA analysis in CD

| | | |
|---|---|---|
| immune response | 118 | 2.35E−39 |
| defense response | 120 | 1.48E−36 |
| response to biotic stimulus | 122 | 4.04E−36 |
| response to pest, pathogen or parasite | 80 | 2.89E−35 |
| response to wounding | 67 | 5.17E−34 |
| response to other organism | 80 | 2.31E−33 |
| response to external stimulus | 74 | 8.73E−33 |
| organisinal physiological process | 159 | 2.97E−29 |
| inflammatory response | 45 | 6.02E−27 |
| response to stress | 97 | 3.16E−26 |
| response to stimulus | 157 | 3.56E−24 |
| cell adhesion | 62 | 2.80E−17 |
| humoral immune response | 29 | 3.78E−15 |
| cell communication | 163 | 5.40E−15 |
| signal transduction | 153 | 1.40E−14 |
| development | 104 | 8.01E−14 |
| organ development | 49 | 1.19E−13 |
| humoral defense mechanism (sensu Vertebrata) | 23 | 5.21E−13 |
| taxis | 23 | 1.75E−12 |
| chemotaxis | 23 | 1.75E−12 |
| cell proliferation | 47 | 2.70E−12 |
| locomotory behavior | 23 | 3.95E−12 |
| antimicrobial humoral response (sensu Vertebrata) | 18 | 9.55E−11 |
| behavior | 26 | 9.63E−11 |
| morphogenesis | 47 | 9.74E−11 |
| antimicrobial humoral response | 18 | 2.01E−10 |
| regulation of cell proliferation | 31 | 4.93E−10 |
| immune cell activation | 19 | 6.66E−10 |
| cell activation | 19 | 7.74E−10 |
| locomotion | 27 | 1.08E−09 |
| localization of cell | 27 | 1.08E−09 |
| cell motility | 27 | 1.08E−09 |
| angiogenesis | 15 | 2.63E−09 |
| blood vessel morphogenesis | 15 | 4.72E−09 |
| blood vessel development | 15 | 4.72E−09 |
| vasculature development | 15 | 4.72E−09 |
| organ morphogenesis | 24 | 1.82E−08 |
| positive regulation of cell proliferation | 19 | 2.10E−08 |
| cell differentiation | 38 | 2.89E−08 |
| negative regulation of biological process | 49 | 3.05E−08 |
| negative regulation of cellular process | 46 | 6.50E−08 |
| response to chemical stimulus | 32 | 6.85E−08 |
| response to abiotic stimulus | 33 | 5.02E−07 |
| phosphate transport | 15 | 6.57E−07 |
| cellular defense response | 15 | 6.57E−07 |
| positive regulation of biological process | 40 | 9.15E−07 |
| lymphocyte activation | 14 | 9.53E−07 |
| regulation of body fluids | 15 | 1.04E−06 |
| hemostasis | 14 | 1.75E−06 |
| cellular morphogenesis | 23 | 4.82E−06 |
| blood coagulation | 13 | 5.24E−06 |
| calcium ion homeostasis | 12 | 5.29E−06 |
| coagulation | 13 | 6.58E−06 |
| death | 36 | 7.09E−06 |
| inorganic anion transport | 17 | 9.76E−06 |
| positive regulation of physiological process | 31 | 1.04E−05 |
| cell surface receptor linked signal transduction | 73 | 1.13E−05 |
| cell death | 35 | 1.53E−05 |
| wound healing | 13 | 1.54E−05 |
| apoptosis | 34 | 1.73E−05 |
| programmed cell death | 34 | 1.86E−05 |
| di-, tri-valent inorganic cation homeostasis | 13 | 3.32E−05 |
| positive regulation of cellular physiological process | 29 | 3.47E−05 |
| negative regulation of physiological process | 37 | 4.75E−05 |
| positive regulation of cellular process | 32 | 5.00E−05 |
| regulation of immune response | 11 | 5.77E−05 |
| regulation of development | 11 | 5.77E−05 |
| metal ion homeostasis | 13 | 6.66E−05 |
| skeletal development | 14 | 6.89E−05 |
| homeostasis | 17 | 8.32E−05 |
| anion transport | 17 | 9.36E−05 |
| cation homeostasis | 13 | 1.17E−04 |
| negative regulation of cellular physiological process | 35 | 1.17E−04 |
| cell-cell signaling | 31 | 1.19E−04 |
| intracellular signaling cascade | 52 | 1.20E−04 |
| regulation of organismal physiological process | 14 | 1.51E−04 |
| hemopoietic or lyraphoid organ development | 11 | 1.54E−04 |
| cell ion homeostasis | 13 | 1.82E−04 |
| negative regulation of cell proliferation | 14 | 2.40E−04 |
| cell migration | 11 | 4.22E−04 |
| ion homeostasis | 13 | 5.28E−04 |
| cell homeostasis | 13 | 0.00100076 |
| regulation of apoptosis | 21 | 0.001371422 |
| regulation of programmed cell death | 21 | 0.001460683 |
| protein kinase cascade | 18 | 0.001541729 |
| tissue development | 13 | 0.004845121 |
| negative regulation of apoptosis | 11 | 0.004904162 |
| negative regulation of programmed cell death | 11 | 0.005147377 |
| second-messenger-mediated signaling | 13 | 0.005699655 |
| cell growth | 11 | 0.009249997 |
| regulation of cell size | 11 | 0.009249997 |

GO analysis of downregulated DE probe sets
between R and NR from LIMMA analysis in IBD

| | | |
|---|---|---|
| immune response | 114 | 2.51E−40 |
| defense response | 117 | 2.10E−38 |
| response to biotic stimulus | 119 | 4.73E−38 |
| response to pest, pathogen or parasite | 78 | 3.04E−36 |
| response to other organism | 79 | 3.15E−35 |
| response to wounding | 65 | 2.07E−34 |
| organismal physiological process | 157 | 4.10E−33 |
| response to external stimulus | 71 | 1.17E−32 |
| response to stress | 95 | 6.31E−28 |
| inflammatory response | 44 | 1.96E−27 |
| response to stimulus | 153 | 1.06E−26 |
| humoral immune response | 32 | 4.56E−19 |
| taxis | 26 | 2.94E−16 |
| chemotaxis | 26 | 2.94E−16 |
| locomotory behavior | 26 | 8.79E−16 |
| cell communication | 154 | 1.91E−15 |
| humoral defense mechanism (sensu Vertebrata) | 24 | 9.33E−15 |
| signal transduction | 141 | 1.28E−13 |
| behavior | 27 | 2.44E−12 |
| cell adhesion | 51 | 2.58E−12 |
| development | 91 | 5.92E−11 |
| organ development | 42 | 8.25E−11 |
| cell proliferation | 42 | 1.15E−10 |
| antimicrobial humoral response (sensu Vertebrata) | 17 | 2.57E−10 |
| antimicrobial humoral response | 17 | 5.17E−10 |
| response to chemical stimulus | 34 | 6.51E−10 |
| negative regulation of biological process | 48 | 5.72E−09 |
| immune cell activation | 17 | 1.04E−08 |
| cell activation | 17 | 1.19E−08 |
| negative regulation of cellular process | 45 | 1.47E−08 |
| response to abiotic stimulus | 34 | 2.17E−08 |
| phosphate transport | 16 | 3.42E−08 |
| cell-cell signaling | 36 | 1.49E−07 |
| morphogenesis | 38 | 2.30E−07 |
| cellular defense response | 15 | 2.37E−07 |
| regulation of cell proliferation | 24 | 1.42E−06 |
| regulation of body fluids | 14 | 2.33E−06 |
| calcium ion homeostasis | 12 | 2.35E−06 |
| lymphocyte activation | 13 | 2.47E−06 |
| hemostasis | 13 | 4.32E−06 |
| positive regulation of biological process | 36 | 5.64E−06 |
| innate immune response | 11 | 5.95E−06 |
| positive regulation of cell proliferation | 15 | 6.07E−06 |
| wound healing | 13 | 6.59E−06 |
| homeostasis | 18 | 7.78E−06 |
| blood coagulation | 12 | 1.37E−05 |
| di-, tri-valent inorganic cation homeostasis | 13 | 1.44E−05 |
| cell surface receptor linked signal transduction | 68 | 1.48E−05 |
| inorganic anion transport | 16 | 1.48E−05 |
| coagulation | 12 | 1.68E−05 |
| negative regulation of physiological process | 36 | 1.87E−05 |
| cell differentiation | 30 | 2.11E−05 |
| locomotion | 19 | 2.35E−05 |
| localization of cell | 19 | 2.35E−05 |
| cell motility | 19 | 2.35E−05 |
| intracellular signaling cascade | 51 | 2.61E−05 |
| regulation of immune response | 11 | 2.83E−05 |
| regulation of development | 11 | 2.83E−05 |
| skeletal development | 14 | 2.89E−05 |
| metal ion homeostasis | 13 | 2.94E−05 |
| death | 32 | 4.80E−05 |
| negative regulation of cellular physiological process | 34 | 5.19E−05 |
| cation homeostasis | 13 | 5.22E−05 |
| tissue development | 16 | 6.65E−05 |
| protein tanase cascade | 20 | 7.02E−05 |
| hemopoietic or lymphoid organ development | 11 | 7.69E−05 |
| cell ion homeostasis | 13 | 8.25E−05 |
| cell death | 31 | 1.01E−04 |
| apoptosis | 30 | 1.23E−04 |
| anion transport | 16 | 1.25E−04 |
| programmed cell death | 30 | 1.31E−04 |
| auti-apoptosis | 12 | 2.31E−04 |
| ion homeostasis | 13 | 2.46E−04 |
| regulation of organismal physiological process | 13 | 2.62E−04 |
| positive regulation of physiological process | 26 | 2.65E−04 |
| organ morphogenesis | 16 | 3.02E−04 |
| positive regulation of cellular physiological process | 25 | 3.51E−04 |
| positive regulation of cellular process | 28 | 3.59E−04 |
| cellular morphogenesis | 18 | 4.62E−04 |
| cell homeostasis | 13 | 4.78E−04 |
| negative regulation of apoptosis | 12 | 7.76E−04 |
| negative regulation of programmed cell death | 12 | 8.22E−04 |
| sensory perception | 32 | 0.001542761 |
| regulation of kinase activity | 11 | 0.001773554 |
| regulation of protein kinase activity | 11 | 0.001773554 |
| regulation of transferase activity | 11 | 0.001873001 |
| second-messenger-mediated signaling | 13 | 0.002922318 |
| regulation of apoptosis | 19 | 0.002964354 |
| regulation of programmed cell death | 19 | 0.003115078 |
| regulation of enzyme activity | 15 | 0.003480013 |
| negative regulation of cell proliferation | 11 | 0.00457427 |
| growth | 12 | 0.008354378 |

SUPPLEMENTARY TABLE 3
The subset of probe sets, identified by PAM analysis in UC, CD, and IBD. The scores indicate whether the probe set is
up- or downregulated in the two classes of samples. The probe sets are ranked based on the decreased predictive value.

| | Probe Set ID | Gene Symbol | Gene Title | NR-score | R-score |
|---|---|---|---|---|---|
| UC (R = 8, | | | | | |
| NR = 16) | | | | | |
| 1 | 206172_at | IL-13RA2 | interleukin 13 receptor, alpha 2 | 0.2108 | −0.4215 |
| 2 | 205680_at | MMP10 | matrix metallopeptidase 10 (stromelysin 2) | 0.0749 | −0.1497 |
| 3 | 206336_at | CXCL6 | chemokine (C-X-C motif) ligand 6 (granulocyte | 0.0687 | −0.1374 |
| | | | chemotactic protein 2) | | |
| 4 | 231227_at | — | Transcribed locus | 0.0629 | −0.1258 |
| 5 | 206953_s_at | LPHN2 | latrophilin 2 | 0.0614 | −0.1228 |
| 6 | 227140_at | — | CDNA FLJ11041 fis, clone PLACE1004405 | 0.0613 | −0.1226 |
| 7 | 205990_s_at | WNT5A | wingless-type MMTV integration site family, member 5A | 0.0574 | −0.1147 |
| 8 | 206924_at | IL-11 | interleukin 11 | 0.0556 | −0.1112 |
| 9 | 212526_at | SPG20 | spastic paraplegia 20 (Troyer syndrome) | 0.0412 | −0.0824 |
| 10 | 213338_at | TMEM158 | transmembrane protein 158 | 0.0382 | −0.0764 |
| 11 | 211959_at | IGFBP5 | insulin-like growth factor binding protein 5 | 0.038 | −0.0759 |
| 12 | 204597_x_at | STC1 | stanniocalcin 1 | 0.0373 | −0.0747 |
| 13 | 204933_s_at | TNFRSF11B | tumor necrosis factor receptor superfamily, | 0.0357 | −0.0714 |
| | | | member 11b (osteoprotegerin) | | |
| 14 | 204222_s_at | GLIPR1 | GLI pathogenesis-related 1 (glioma) | 0.0332 | −0.0664 |
| 15 | 227361_at | HS3ST3B1 | heparan sulfate (glucosamine) 3-O-sulfotransferase 3B1 | 0.029 | −0.058 |
| 16 | 203424_s_at | IGFBP5 | insulin-like growth factor binding protein 5 | 0.0228 | −0.0457 |
| 17 | 211671_s_at | NR3C1 | nuclear receptor subfamily 3, group C, member 1 | 0.0221 | −0.0441 |
| | | | (glucocorticoid receptor) | | |
| 18 | 201645_at | TNC | tenascin C (hexabrachion) | 0.0211 | −0.0422 |
| 19 | 230746_s_at | STC1 | Stanniocalcin 1 | 0.0187 | −0.0374 |
| 20 | 228128_x_at | PAPPA | pregnancy-associated plasma protein A, pappalysin 1 | 0.0187 | −0.0374 |
| 21 | 203603_s_at | ZEB2 | zinc finger E-box binding homeobox 2 | 0.0185 | −0.0369 |
| 22 | 209795_at | CD69 | CD69 molecule | 0.018 | −0.0359 |
| 23 | 206623_at | PDE6A | phosphodiesterase 6A, cGMP-specific, rod, alpha | −0.0166 | 0.0332 |
| 24 | 212977_at | CXCR7 | chemokine (C-X-C motif) receptor 7 | 0.016 | −0.0319 |
| 25 | 203887_s_at | THBD | thrombomodulin | 0.0153 | −0.0306 |
| 26 | 202422_s_at | ACSL4 | acyl-CoA synthetase long-chain family member 4 | 0.0121 | −0.0243 |
| 27 | 203680_at | PRKAR2B | protein kinase, cAMP-dependent, regulatory, type II, beta | 0.0118 | −0.0236 |
| 28 | 1555638_a_at | SAMSN1 | SAM domain, SH3 domain and nuclear localization signals 1 | 0.011 | −0.0219 |
| 29 | 226001_at | KLHL5 | kelch-like 5 (Drosophila) | 0.0084 | −0.0169 |
| 30 | 205207_at | IL6 | interleukin 6 (interferon, beta 2) | 0.0064 | −0.0127 |
| 31 | 207610_s_at | EMR2 | egf-like module containing, mucin-like, hormone receptor-like 2 | 0.0062 | −0.0125 |
| 32 | 205443_at | SNAPC1 | small nuclear RNA activating complex, polypeptide 1, 43 kDa | 0.0059 | −0.0118 |
| 33 | 205828_at | MMP3 | matrix metallopeptidase 3 (stromelysin 1, progelatinase) | 0.0046 | −0.0092 |
| 34 | 1555229_a_at | C1S | complement component 1, s subcomponent | 0.0041 | −0.0083 |
| 35 | 204932_at | TNFRSF11B | tumor necrosis factor receptor superfamily, member 11b | 0.0036 | −0.0072 |
| | | | (osteoprotegerin) | | |
| 36 | 214247_s_at | DKK3 | dickkopf homolog 3 (Xenopus laevis) | 0.0029 | −0.0058 |
| 37 | 209960_at | HGF | hepatocyte growth factor (hepapoietin A; scatter factor) | 0.0026 | −0.0053 |
| CD (R = 12, | | | | | |
| NR = 7) | | | | | |
| 1 | 206025_s_at | TNFAIP6 | tumor necrosis factor, alpha-induced protein 6 | 0.7572 | −0.4417 |
| 2 | 206026_s_at | TNFAIP6 | tumor necrosis factor, alpha-induced protein 6 | 0.4848 | −0.2828 |
| 3 | 206924_at | IL-11 | interleukin 11 | 0.4385 | −0.2558 |
| 4 | 213524_s_at | G0S2 | G0/G1 switch 2 | 0.3358 | −0.1959 |
| 5 | 214370_at | S100A8 | S100 calcium binding protein A8 | 0.2959 | −0.1726 |
| 6 | 205863_at | S100A12 | S100 calcium binding protein A12 | 0.2852 | −0.1664 |
| 7 | 203535_at | S100A9 | S100 calcium binding protein A9 | 0.2219 | −0.1294 |
| 8 | 205681_at | BCL2A1 | BCL2-related protein A1 | 0.1908 | −0.1113 |
| 9 | 232629_at | PROK2 | prokineticin 2 | 0.1904 | −0.1111 |
| 10 | 205207_at | IL6 | interleukin 6 (interferon, beta 2) | 0.1646 | −0.096 |
| 11 | 204959_at | MNDA | myeloid cell nuclear differentiation antigen | 0.1167 | −0.0681 |
| 12 | 222088_s_at | SLC2A14 /// | solute carrier family 2 (facilitated glucose | 0.108 | −0.063 |
| | | SLC2A3 | transporter), member 3 /// solute carrier family 2 | | |
| | | | (facilitated glucose transporter), member 14 | | |
| 13 | 206172_at | IL-13RA2 | interleukin 13 receptor, alpha 2 | 0.1053 | −0.0614 |
| 14 | 202917_s_at | S100A8 | S100 calcium binding protein A8 | 0.1015 | −0.0592 |
| 15 | 229947_at | PI15 | peptidase inhibitor 15 | 0.0805 | −0.047 |
| 16 | 205119_s_at | FPR1 | formyl peptide receptor 1 | 0.0775 | −0.0452 |
| 17 | 1554997_a_at | PTGS2 | prostaglandin-endoperoxide synthase 2 | 0.0445 | −0.0259 |
| | | | (prostaglandin G/H synthase and cyclooxygenase) | | |
| 18 | 202499_s_at | SLC2A3 | solute carrier family 2 (facilitated glucose transporter), member 3 | 0.0318 | −0.0185 |
| 19 | 205568_at | AQP9 | aquaporin 9 | 0.0252 | −0.0147 |
| 20 | 229723_at | TAGAP | T-cell activation GTPase activating protein | 0.024 | −0.014 |
| | | | neutrophil cytosolic factor 2 (65 kDa, chronic granulomatous | | |
| 21 | 209949_at | NCF2 | disease, autosomal 2) | 0.016 | −0.0094 |
| IBD (R = 20, | | | | | |
| NR = 23) | | | | | |
| 1 | 206172_at | IL-13RA2 | interleukin 13 receptor, alpha 2 | 0.1886 | −0.2169 |
| 2 | 206924_at | IL-11 | interleukin 11 | 0.016 | −0.0184 |

1. An in vitro method of determining if a subject suffering from an inflammatory condition of the large intestine and/or small intestine will respond to anti-TNFα therapy, wherein the method comprises:
obtaining a biological sample from the subject;
analyzing the level of it IL-13R(alpha)2 expression or activity of expression product of IL-13R(alpha)2 in the biological sample, and
comparing said level of expression or activity with the 13ralpha2 expression or activity from a control sample;
wherein a different level of IL-13R(alpha)2 expression or activity relative to the control sample is an indication of response to anti-TNFα therapy or a propensity thereto in the subject.

2. The in vitro method of claim 1, wherein a decreased level of IL-13R(alpha)2 in comparison to the control sample is indicative of a positive response to the anti-TNFα therapy in the subject.

3. The in vitro method according to claim 1, wherein the inflammatory condition of the large intestine and/or small intestine is an inflammatory bowel disease.

4. The in vitro method according to claim 1, further comprising predicting if the subject will respond to anti-TNFα therapy for Crohn's disease.

5. The in vitro method according to claim 1, further comprising predicting if the subject will respond to an anti-TNFα antibody therapy that blocks action of TNFα by preventing TNFα from binding to its receptor in a cell.

6. The in vitro method of claim 5, wherein a decreased level of IL-13R(alpha)2 is indicative of a positive response thereto and is predictive of a responder.

7. The in vitro method according to claim 3, further comprising predicting if the subject suffering from an inflammatory bowel disease will respond to an anti-TNFα antibody therapy that blocks the action of TNFα by preventing TNFα from binding to its receptor in a cell.

8. The in vitro method of claim 7, wherein a decreased level of IL-13R(alpha)2 is indicative of a positive response thereto and is indicative of a responder.

9. The in vitro method according to claim 1, wherein the subject is on anti-TNFα therapy for an inflammatory bowel disease (“IBD”), or has a propensity to IBD, said method further comprising:
obtaining an expression profile in a biological sample isolated from the subject, wherein said expression profile consists of the analysis of the level of IL-13R(alpha)2 expression or activity of an IL-13R(alpha)2 expression product in combination with gene expression level or activity of a gene product of at least one gene selected from the group consisting of TNFRSF11B, STC1, PTGS2 and IL-11; and
comparing said obtained expression profile to a reference expression profile to determine whether said biological sample is from a subject having an IBD phenotype or a propensity thereto.

10. The in vitro method of claim 9 wherein the expression profile consists of any one of the following combinations:
IL-13R(alpha)2 and TNFRSF11B;
IL-13R(alpha)2 and STC1;
IL-13R(alpha)2 and PTGS2;
IL-13R(alpha)2 and IL-11;
IL-13R(alpha)2 and STC1 and PTGS2;
IL-13R(alpha)2 and TNFRSF11B and PTGS2;
IL-13R(alpha)2 and TNFRSF11B and STC1;
IL-13R(alpha)2 and IL-11 and TNFRSF11B;
IL-13R(alpha)2 and IL-11 and STC1;
IL-13R(alpha)2 and IL-11 and PTGS2;
IL-13R(alpha)2 and TNFRSF11B and PTGS2 and STC1;
IL-13R(alpha)2 and IL-11 and PTGS2 and STC1;
IL-13R(alpha)2 and TNFRSF11B and IL-11 and STC1; or
IL-13R(alpha)2 and TNFRSF11B and PTGS2 and IL-11.

11. The in vitro method according to claim 1, to predict the response or non-response of a subject on an anti-TNFα treatment of inflammatory bowel disease, or a propensity thereto, said method further comprising:
(a) obtaining an expression profile in a biological sample isolated from the subject, wherein said expression profile consists of the analysis of the level of IL-13R(alpha)2 expression or activity of an IL-13R(alpha)2 expression product in combination with the gene expression level or activity of a gene product of at least two gene selected from the group consisting of TNFRSF11B, STC1, PTGS2, and IL-11; and
(b) comparing said obtained expression profile to a reference expression profile to determine whether said sample is from a subject having a inflammatory bowel disease phenotype or a propensity thereto.

12. The in vitro method according to claim 3, to predict the response or non-response of a subject on an anti-TNFα treatment of inflammatory bowel disease, or a propensity thereto, said method further comprising:
(a) obtaining an expression profile in a biological sample isolated from the subject, wherein said expression profile consists of the analysis of the level of IL-13R(alpha)2 expression or activity of an IL-13R(alpha)2 expression product in combination with the gene expression level or activity of a gene product of at least three gene selected from the group consisting of TNFRSF11B, STC1, PTGS2, and IL-11; and (b) comparing said obtained expression profile to a reference expression profile to determine whether said sample is from subject having a IBD phenotype or a propensity thereto.

13. The in vitro method according to claim 3, to predict the response or non-response of a subject on an anti-TNFα treatment of inflammatory bowel disease, or having a propensity thereto, said method comprising:
obtaining an expression profile in a biological sample isolated from the subject, wherein said expression profile consists of analyzing the level of IL-13R(alpha)2 expression or activity of an expression product of at the gene cluster of the genes IL-13R(alpha)2, TNFRSF11B, STC1, PTGS2, and IL-11; and
comparing said obtained expression profile to a reference expression profile to determine whether said sample is from a subject having an IBD phenotype or a propensity to IBD.

14. The in vitro method according to claim 1, wherein the expression product is a nucleic acid molecule selected from the group consisting of mRNA and cDNA mRNA or polypeptides derived therefrom.

15. The in vitro method according to claim 1, wherein the sample isolated form the subject is from a colonic mucosal biopsy.

16. The in-vitro method according to claim 1, comprising the detection of the level of the nucleic acids or polypeptides carried out utilizing at least one binding agent specifically binding to the nucleic acids or polypeptides to be detected.

17. The in-vitro method according to claim 1, wherein the binding agent is detectably labelled.

18. The in-vitro method according to claim 17, wherein the label is selected from the group consisting of a radioisotope, a bioluminescent compound, a chemiluminescent compound, a fluorescent compound, a metal chelate, biotin, digoxigenin, and an enzyme.

19. The in-vitro method according to according to claim 1, wherein at least one binding agent is an aptamer or an antibody selected from the group consisting of a monoclonal antibody; a polyclonal antibody; a fab-fragment; a single chain antibody; and an antibody variable domain sequence.

20. The in-vitro method according to claim 16, with at least one binding agent being a nucleic acid hybridising to a nucleic acid utilized for the detection of marker molecules, IL-13R(alpha)2, TNFRSF11B, STC1, PTGS2, and IL-11 expression

21. The in-vitro method of claim 20, wherein the detection reaction comprises a nucleic acid amplification reaction.

22. The in-vitro method of claim 21, the method wherein the method is be utilized for in-situ detection

23. A diagnostic test kit for use in diagnosing a subject for responsiveness to an anti-TNFα treatment of inflammatory bowel disease and/or Crohn's disease (cd), or for use in monitoring the effectiveness of therapy of inflammatory bowel disease in patients receiving an anti-TNFα therapy, the diagnostic kit comprising:
a predetermined amount of an antibody specific for IL-13R(alpha)2;
a predetermined amount of a specific binding partner to said antibody;
buffers and other reagents necessary for monitoring detection of antibody bound to IL-13R(alpha)2; and wherein either said antibody or said specific binding partner is detectably labelled.

24. (canceled)

25. The diagnostic test kit of claim 23 comprising:
a predetermined amount of two different antibodies each specific for two different proteins of the group IL-13R(alpha)2, TNFRSF11B, STC1, PTGS2, and IL-11;
a predetermined amount of a specific binding partner to said antibodies;
buffers and other reagents necessary for monitoring detection of antibody bound to the selected proteins of the group consisting of IL-13R(alpha)2, TNFRSF11B, STC1, PTGS2, and IL-11; and wherein either said antibody or said specific binding partner is detectably labelled.

26. (canceled)

27. The diagnostic test kit of claim 23, comprising:
a predetermined amount of a predetermined amount of three different antibodies each specific for three different proteins of the group consisting of IL-13R(alpha)2, TNFRSF11B, STC1, PTGS2, and IL-11;
a predetermined amount of a specific binding partner to said antibodies;
buffers and other reagents necessary for monitoring detection of antibody bound to the selected proteins of the IL-13R(alpha)2, TNFRSF11B, STC1, PTGS2, and IL-11; and wherein either said antibody or said specific binding partner is detectably labelled.

28. (canceled)

29. The diagnostic test kit of claim 23, comprising:
a predetermined amount of four different antibodies each specific for four different proteins of the group IL-13R(alpha)2, TNFRSF11B, STC1, PTGS2 and IL-11;
a predetermined amount of a specific binding partner to said antibodies;
buffers and other reagents necessary for monitoring detection of antibody bound to the selected proteins of the group consisting of IL-13R(alpha)2, TNFRSF11B, STC1, PTGS2, and IL-11; and wherein either said antibody or said specific binding partner is detectably labelled.

30. (canceled)

31. The diagnostic test kit of claim 23, comprising:
a predetermined amount of an antibody specific for each of proteins of the group IL-13R(alpha)2, TNFRSF11B, STC1, PTGS2, and IL-11;
a predetermined amount of a specific binding partner to said antibodies;
buffers and other reagents necessary for monitoring detection of antibody bound to the selected proteins of the group consisting of IL-13R(alpha)2, TNFRSF11B, STC1, PTGS2, and IL-11; and wherein either said antibody or said specific binding partner is detectably labelled.

32. (canceled)

33. A diagnostic test kit for use in diagnosing a subject for responsiveness to anti-TNFα treatment of inflammatory bowel disease (ibd) or for use in monitoring the effectiveness of therapy of inflammatory bowel disease in patients receiving an to anti-TNFα therapy, the diagnositic kit comprising:
a) a nucleic acid encoding the IL-13R(alpha)2 protein;
b) reagents useful for monitoring the expression level of the one or more nucleic acids or proteins encoded by the nucleic acids of step a); and
c) instructions for use of the kit.

34. The diagnostic test kit of claim 34, comprising:
a) nucleic acids encoding the IL-13R(alpha)2, TNFRSF11B, STC1, PTGS2, and IL-11 protein;
b) reagents useful for monitoring the expression level of the one or more nucleic acids or proteins encoded by the nucleic acids of step a); and
c) instructions for use of the kit.
